(19) United States
(12) Patent Application Publication
(10) Pub. No.: US 2026/0216274 A1
(43) Pub. Date: Jul. 30, 2026

(54) ADMINISTRATION OF YUNNAN BAIYAO OR XINGNAOJING IN PATIENTS WITH MODERATE-TO-SEVER TRAUMATIC BRAIN INJURY AND CRANIOTOMY

(71) Applicant: Lotus Biotech.com LLC

(72) Inventor: Guoqiang Xing, Gaithersburg, MD (US)

(73) Assignee: Lotus Biotech.com LLC, (US)

(21) Appl. No.: 19/574,365

(22) Filed: Mar. 22, 2026

Related U.S. Application Data
(63) Continuation-in-part of application No. 17/812,171, filed on Jul. 12, 2022, now Pat. No. 12,582,691.
(60) Provisional application No. 63/220,936, filed on Jul. 12, 2021.

(51) Int. Cl. A61K 36/9066; A61K 9/00; A61K 31/045; A61K 35/12; A61K 36/63; A61P 25/00; G01N 33/68

(52) U.S. Cl. CPC A61K 36/9066; A61K 9/0019; A61K 31/045; A61K 35/12; A61K 36/63; A61P 25/00; G01N 33/6896; G01N 2333/4727; G01N 2333/90283; G01N 2800/28

(57) ABSTRACT

A method of treating a patient with moderate-to-severee traumatic brain injury (TBI) undergoing emergency craniotomy includes administering, in addition to orthodox therapy (OT), intravenous Xingnaojing (XNJ) for seven consecutive days in an intensive care setting, wherein acute postoperative neurological recovery is improved as measured by Glasgow Coma Scale (GCS) during postoperative Days 1, 3, 5, and 7, wherein long-term functional outcome is improved as measured by Glasgow Outcome Scale (GOS) and Karnofsky Performance Status (KPS) at 30 and 90 days, and wherein secondary-injury biomarkers are modulated by reducing serum S100B and preserving or restoring serum superoxide dismutase (SOD) activity relative to OT alone.

### CROSS-REFERENCE TO RELATED APPLICATION

This application is a continuation-in-part under 35 U.S.C. § 120 of U.S. patent application Ser. No. 17/812,171, filed on Jul. 12, 2022, which claims the benefit under 35 U.S.C. § 119 of provisional application 63/220,936, filed on Jul. 12, 2021, which is incorporated herein by reference.

### BACKGROUND

One technical field of the present disclosure is methods of treating a brain disorder in a subject's brain. Another technical field is medicinal methods of administering Yunnan Baiyao (YB) or Xingnaojing (XNJ) to improve postoperative recovery and long-term clinical prognosis of patients with moderate-to-severee traumatic brain injury (TBI) and emergency craniotomy.

Secondary brain injury following traumatic brain injury (TBI) is currently the main target for therapeutic intervention. TBI is a complex and diffuse injury with multi-modes of lesions, including focal cerebral contusion, parenchymal laceration, hemorrhages (subdural, epidural, and/or subarachnoid), diffuse axonal injury, diffuse hypoxic-ischemic insults, cerebral swelling, increased intracranial pressure, and brain compression and herniation. Adjacent brain tissues that are not destroyed immediately following the primary injury often experience sub-acute injury or delayed death caused by secondarily generated auto-destructive factors. Blood S100B protein is a glial-derived, brain specific calcium-binding protein that is highly elevated after TBI. High concentration of S100B overproduced by injured astrocytes after TBI exacerbates neurovascular inflammatory responses and apoptosis through interaction with the receptor for advanced glycation end products (RAGE). Serum S100B protein is a reliable biomarker of for assessing the severity of TBI and secondary brain injury. Extracellular Superoxide Dismutase (SOD) is the primary extracellular scavenger responsible for scavenging reactive oxygen species (ROS), which are major contributors to oxidative damage and secondary injury following TBI. Depletion in SOD activity and antioxidant function of the body has been reported in TBI, and is linked to increased risk or poor prognosis of acute injury and organ failure. Although orthodox treatments (OT) such as hyperosmolar therapy, sedation, barbiturate coma, hypothermia therapy, and ventricular drainage have been used in TBI management, they are often not effective. Surgical removal of intracranial hematoma and fractured brain tissue (craniotomy/decompression craniectomy) can reduce the mortality of severe TBI, incidence of ICP, and length of hospital stay in TBI patients (Rutigliano et al., 2006; Shim et al., 2018; Lu et al., 2020). Surgery, however, could also worsen the clinical outcomes of TBI (Qiu et al., 2009; Su et al., 2019), partly due to secondary injury, increased bleeding and inflammatory complications (Muller et al., 1988; Tapper et al., 2017; Cheng et al., 2018; Raso Vasquez et al., 2018; Glass et al., 2019; Lu et al., 2020). Timely appropriate drug treatment after craniotomy could potentially reduce secondary injuries and improve the prognosis of TBI. So far, all experimental drugs for the treatment of TBI failed in clinical trials. Recent evidence suggests that complementary and alternative medicine (CAM) may benefit the clinical outcomes of TBI. Yunnan Baiyao (YB) is a traditional hemostatic drug for wound healing. Until now, its efficacy for postoperative TBI has not been evaluated due to the technical difficulty/complexity, high cost, and high risk associated with clinical trials of TBI. Xingnaojing (XNJ) is an intravenous formula based on the traditional medicine AngongNiuhuang Pill (ANP). To date, the utility and effectiveness of administration of YB and XNJ as the neuroprotectant and anti-neuroinflammation agents in patients with traumatic brain injury (TBI) and craniotomy have remained unexplored. The Glasgow Coma Scale (GCS) is used to evaluate disturbance of consciousness with eye opening, speech condition, and motor response (mild=13-15, moderate=8-12, severe=3-7). The Glasgow Outcome Scale (GOS) and Karnofsky Performance Scale (KPS) are scales used to evaluate long-term clinical prognosis of TBI. Both products are GMP-certified and approved for clinical use in China, with consistent compositions across manufacturing batches.

Secondary brain injury after TBI is a major driver of poor outcomes and remains a key target for intervention. TBI can involve contusion, hemorrhage, diffuse axonal injury, brain swelling, and increased intracranial pressure, and it can trigger delayed cell death through oxidative stress and inflammation. In this CIP, the biomarker endpoints are intentionally limited to two clinically validated measures: (i) serum S100B, a glial-derived protein that rises after brain injury and, at high levels, is associated with inflammatory/apoptotic secondary injury; and (ii) SOD activity, which reflects the body's extracellular anti-oxidative capacity against reactive oxygen species. Accordingly, the claimed therapeutic effects of YB and XNJ in moderate-to-severee TBI with craniotomy are supported by modulation of S100B and preservation/restoration of SOD activity, together with functional endpoints (GCS/GOS/KPS).

### SUMMARY

The present disclosure relates to the neuroprotection and neurotherapies by two complementary and alternative medicines Yunnan Baiyao (also termed “YB”) and Xingnaojing (also termed “XNJ”) in patients with moderate-to-severe traumatic brain injury (TBI) and emergency craniotomy. The present disclosure pertains to the discovery of novel therapies for neurodegeneration and/or neuroinflammation-related brain neuropathies, including spinal cord injury, brain tumor, ischemia stroke, and cerebrohemorrhage, and methods for preventing the secondary-injuries of craniotomy operations on these disorders.

Described herein is a method having many attributes and embodiments including, but not limited to, that set forth or described in this brief summary. It is not intended to be all-inclusive and the claims are not limited to or by the features or embodiments identified in this brief summary.

The present disclosure relates to neuroprotective therapies using Yunnan Baiyao (“YB”) and Xingnaojing (“XNJ”) in patients with moderate-to-severee traumatic brain injury (TBI) undergoing emergency craniotomy. The disclosed utility is specifically directed to the peri-craniotomy acute phase of TBI, where oxidative stress and inflammation contribute to secondary injury. The invention is not directed to conventional stroke indications; rather, it claims a distinct clinical context (moderate-to-severee TBI with craniotomy) and a defined outcome framework using S100B and SOD biomarkers coupled to functional recovery endpoints.

Accordingly, in one aspect, the present disclosure is directed to the novel and significant inhibitory activities of orally administered or stomach-tube administered YB, on TBI-induced increase in blood S100B level during the acute phase of postoperative TBI, demonstrating a significant neuroprotection and anti-neuroinflammation activities of YB against secondary brain injuries in patients with moderate-to-severe TBI and craniotomy.

In another aspect, the present disclosure is directed to the novel and significant protection against TBI-induced depletion of blood SOD activity during the acute phase of moderate-to-severe TBI and craniotomy by orally-administered or stomach tube-administered YB. SOD is a biomarker of body oxidative potential against TBI-induced damage.

In another aspect, the present disclosure is directed to the novel and significant inhibitory activities of intravenously-administered XNJ on TBI-induced increase in blood S100B level during the acute phase of postoperative TBI, demonstrating a significant neuroprotection and anti-neuroinflammation activity of XNJ against secondary brain injuries in patients with moderate-to-severe TBI and craniotomy.

In another aspect, the present disclosure is directed to the novel and significant protecting activity of intravenously-administered XNJ against TBI-induced depletion of blood SOD activity and therefore, XNJ preserved body anti-oxidative potential against TBI-induced oxidative damage during the acute phase of moderate-to-severe TBI and craniotomy.

In another aspect, the present disclosure is directed to the significant improvement in GCS score and therefore a more rapid recovery from coma by orally- or stomach tube-administered YB during the acute phase of TBI in patients with moderate-to-severe TBI and craniotomy.

In another aspect, the present disclosure is directed to the significant improvement in GCS score and therefore a more rapid recovery from coma after intravenous administration of XNJ during the acute phase of TBI in patients with moderate-to-severe TBI and craniotomy.

In another aspect, the present disclosure is directed to the significant improvement in 30- and 90-days Glasgow Outcome Scale (GOS) and Karnofsky Performance Scale (KPS) in TBI patients who received YB administrations during the acute phase of TBI, therefore, demonstrating improved long-term recovery in physical strength, functional rehabilitation and the abilities of daily life and work by YB.

In another aspect, the present disclosure is directed to the significant improvement in Glasgow Outcome Scale (GOS) and Karnofsky Performance Scale (KPS) at 30-days and 90-days post craniotomy in TBI patients who received intravenous XNJ during the acute phase of TBI, therefore, demonstrating improved and sustained long-term recovery by XNJ in physical strength, functional rehabilitation and the abilities of daily life and work.

In another aspect, the present disclosure is directed to the significant correlations between the acute phase GCS scores and chronic phase GOS/KPS scores in pooled TBI patients, therefore, demonstrating that a more rapid recovery during the acute phase of TBI is a predictor of more favorable long-term clinical prognosis of patients with TBI and craniotomy.

In another aspect, the present disclosure is directed to the findings that serum S100B levels were negatively correlated with GCS/GOS/KPS scores in patients with moderate-to-severe TBI and craniotomy. Because GCS reflects acute phase recovery, whereas GOS/KPS reflects long-term prognosis, reduced serum S100B during the acute phase of TBI in patients administered with YB or XNJ would predict more favorable clinical outcomes of both short-term and long-term. This study also demonstrates that a higher S100B level at admission or at postoperative day 7 was a predictor of poor TBI outcomes (low GCS/GOS/KPS scores).

In another aspect, the present disclosure is directed to the findings that serum S100B levels were negatively correlated with GCS/GOS/KPS scores in pooled patients and in different treatment groups. Because GCS predict acute phase recovery whereas GOS/KPS predict long-term prognosis, the reduced serum S100B during the acute phase of TBI in patients administered with YB or XNJ would predict their long-term clinical outcome. This study demonstrated that serum S100B levels either on admission day or on postoperative day could be a good biomarker in evaluating the therapeutic effects of novel therapies in the outcomes of TBI.

In one aspect, the present disclosure is directed to serum SOD activity which was positively correlated with GCS/GOS/KPS scores (p<0.01) in the pooled and in the 3 treatment groups, but not in the OT group. Serum SOD level on postoperative Day 7 was a better predictor of GCS/GOS/KPS scores than SOD measured on other days in the pooled and 3 adjuvant treatment groups but not in the OT group, demonstrating that a high level of serum SOD activity was a positive predictor of TBI clinical prognosis.

In one aspect, the present disclosure directs to the similar therapeutic effects between the two different doses of YB (1 or 2 g/day) and the intravenous dose of XNJ (20 ml/day) in patients with moderate-to-severe TBI and craniotomy, demonstrating a lower dose of YBY (1 g/day) was adequate to generate most of the beneficial effects of the higher dose of YB (2 g/day) or the intravenous XNJ (20 ml/day). Whereas intravenous XNJ would be an option for TBI patients in a coma.

### BRIEF DESCRIPTION OF THE DRAWINGS

FIG. 1 is the flow diagram of the patient enrollment, intervention, and follow up.

FIG. 2 is the repeated-measure analysis demonstrating significant and different impacts of 7-day treatment of orthodox therapy (OT), intravenous Xingnaojing (XNJ), low-dose Yunnan Baiyao (1-YB, 1,000 mg/day), or high-dose Yunnan Baiyao (h-YB, 2,000 mg/day) on: (A) Glasgow Coma Scale (GCS); (B) Serum S100B concentration; (C) Serum superoxide dismutase (SOD) activity; (D) Glasgow Outcome Scale (GOS) and; (E) Karnofsky Performance Scale (KPS), in patients with acute moderate-to-severee TBI and emergency craniotomy.

FIG. 3A, FIG. 3B, FIG. 3C, FIG. 3D, FIG. 3E, FIG. 3F, FIG. 3G, FIG. 3H, FIG. 3I show significant differences in GCS, serum S100B and SOD between the 4 treatment groups; specifically, FIG. 3A shows that GCS score was lowest on admission in all groups but improved more quickly and became significantly greater in XNJ, L-YB, h-YB groups than in OT group on Day 3, 5 and 7 (p<0.01); FIG. 3B shows the improvement in GCS and; FIG. 3C shows that rate of improvement (%) in GCS were significantly greater in XNJ, L-YB, h-YB groups than in OT group after 3, 5 and 7 days of emergency craniotomy (p<0.01) and greater in h-YB than in L-YB and XNJ groups after 3, 5 and 7 days of emergency craniotomy (p<0.05, all); FIG. 3D shows that S100B concentration increased quickly after admission and peaked in all groups on Days 3 before it fell more rapidly and became significantly lower (p<0.01) in the XNJ, L-YB and h-YB groups than in the OT group after 3, 5 and 7 days of emergency craniotomy; FIG. 3E and FIG. 3F show that increases and percentage increase in serum S100B was significantly greater (p<0.01) in OT than in XNJ and h-YB groups on Day 3 whereas reduction and reduction rate was significantly greater in XNJ, L-YB and h-YB groups than in OT group after 5 and 7 days of emergency craniotomy; FIG. 3G, FIG. 3H, FIG. 3I show serum SOD activity declined rapidly after craniotomy and reached nadir on Days 3 day in all groups with a greater reduction found in OT group (38.3%) than in XNJ, L-YB and h-YB groups (16.7%, 23.4% and 20.7%) before increased more rapidly and became significantly higher in the XNJ, L-YB, and h-YB groups than in the OT group after 3, 5, and 7 days of emergency craniotomy (p<0.01). By Day 7, XNJ, L-YB and h-YB groups resumed more of the baseline SOD activities (99.5%, 91.8% and 94.8%, respectively) than the OT group (69.2%) (p<0.01). *, p<0.05, **p<0.01 vs. OT; a, p<0.05, aa, p<0.01 vs. XNJ; b, p<0.05, bb, p<0.01 vs. L-YB.

### DETAILED DESCRIPTION

In the following description, for the purposes of explanation, numerous specific details are set forth in order to provide a thorough understanding of the present invention. It will be apparent, however, that the present invention may be practiced without these specific details. In other instances, well-known structures and devices are shown in block diagram form in order to avoid unnecessarily obscuring the present invention.

The text of this disclosure, in combination with the drawing figures, is intended to state in prose the algorithms that are necessary to program a computer to implement the claimed inventions, at the same level of detail that is used by people of skill in the arts to which this disclosure pertains to communicate with one another concerning functions to be programmed, inputs, transformations, outputs and other aspects of programming. That is, the level of detail set forth in this disclosure is the same level of detail that persons of skill in the art normally use to communicate with one another to express algorithms to be programmed or the structure and function of programs to implement the inventions claimed herein.

One or more different inventions may be described in this disclosure, with alternative embodiments to illustrate examples. Other embodiments may be utilized and structural, logical, software, electrical and other changes may be made without departing from the scope of the particular inventions. Various modifications and alterations are possible and expected. Some features of one or more of the inventions may be described with reference to one or more particular embodiments or drawing figures, but such features are not limited to usage in the one or more particular embodiments or figures with reference to which they are described. Thus, the present disclosure is neither a literal description of all embodiments of one or more of the inventions nor a listing of features of one or more of the inventions that must be present in all embodiments.

Headings of sections and the title are provided for convenience but are not intended as limiting the disclosure in any way or as a basis of interpreting the claims. Devices that are described as in communication with each other need not be in continuous communication with each other, unless expressly specified otherwise. In addition, devices that are in communication with each other may communicate directly or indirectly through one or more intermediaries, logical or physical.

A description of an embodiment with several components in communication with one other does not imply that all such components are required. Optional components may be described to illustrate a variety of possible embodiments and to more fully illustrate one or more aspects of the inventions. Similarly, although process steps, method steps, algorithms or the like may be described in a sequential order, such processes, methods and algorithms may generally be configured to work in different orders, unless specifically stated to the contrary. Any sequence or order of steps described in this disclosure is not a required sequence or order. The steps of described processes may be performed in any order practical. Further, some steps may be performed simultaneously. The illustration of a process in a drawing does not exclude variations and modifications, does not imply that the process or any of its steps are necessary to one or more of the invention(s), and does not imply that the illustrated process is preferred. The steps may be described once per embodiment, but need not occur only once. Some steps may be omitted in some embodiments or some occurrences, or some steps may be executed more than once in a given embodiment or occurrence. When a single device or article is described, more than one device or article may be used in place of a single device or article. Where more than one device or article is described, a single device or article may be used in place of the more than one device or article.

The functionality or the features of a device may be alternatively embodied by one or more other devices that are not explicitly described as having such functionality or features. Thus, other embodiments of one or more of the inventions need not include the device itself. Techniques and mechanisms described or referenced herein will sometimes be described in singular form for clarity. However, it should be noted that particular embodiments include multiple iterations of a technique or multiple manifestations of a mechanism unless noted otherwise. Process descriptions or blocks in figures should be understood as representing modules, segments, or portions of code which include one or more executable instructions for implementing specific logical functions or steps in the process. Alternate implementations are included within the scope of embodiments of the present invention in which, for example, functions may be executed out of order from that shown or discussed, including substantially concurrently or in reverse order, depending on the functionality involved.

The present disclosure involves the clinic exploration and validation of the therapeutic effects of orally-administered high and low doses ((1 or 2 g/day) of YB and intravenously-administered XNJ (20 ml/day) during the acute phase of TBI for 7 days in comparison with those of OT intervention alone, on post-surgery recovery, secondary brain injury, long-term clinical prognosis and functional recovery of patients with moderate-to-severee TBI and emergency craniotomy, based on the differences and postoperative changes in Glasgow Coma Scale (GCS), serum S100B level, serum SOD activity, Glasgow Outcome Scale (GOS), and Karnofsky Performance Scale (KPS) between the four treatment groups (OT, h-YB, 1-YB and XNJ) measured at baseline, 7-, 30-, and 90-days post-surgery, respectively.

The present disclosure involves the exploration of the effectiveness of YB and XNJ administration on the outcome of secondary brain injury measured by changes in serum levels of S100B and superoxide dismutase (SOD) activity. Both YB and XNJ therapies improved the acute recovery and long-term clinical prognosis of TBI and craniotomy, in part, by protecting glial cells from TBI-induced S100B overexpression and the associated secondary injury and by preserving and more rapidly restoring lost body SOD antioxidative after TBI. S100B is a brain- and glial-specific calcium-binding protein that promotes neuronal survival at low concentration but exacerbates neurovascular inflammatory responses and apoptosis at high concentration induced after TBI. Serum S100B protein is a validated biomarker of TBI severity. Others reported that postoperative serum S100B levels predicted ongoing brain damage after meningioma surgery. Elevated serum S100B is a predictive index of overall injury and prognosis after trauma/surgery. Extracellular superoxide dismutase (SOD) is the only extracellular scavenger of superoxide anion (O₂⁻) and radicals, which are major causes of oxidative damage and secondary injury of TBI. Serum SOD activity reflects the body's defense capability against free radicals-induced neural damage. Massive production and release of superoxide and radicals after TBI can cause artery dilation, edema, and hemostasis whereas SOD can dose-dependently scavenge excess superoxide anion, reduce brain edema, and attenuate acute brain injury after subarachnoid hemorrhage.

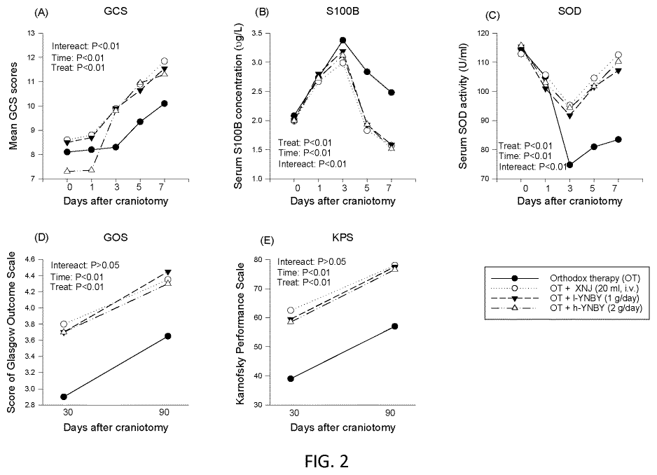

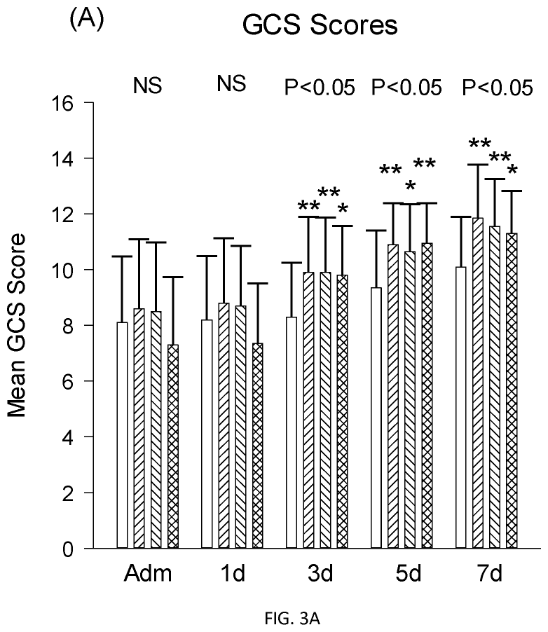

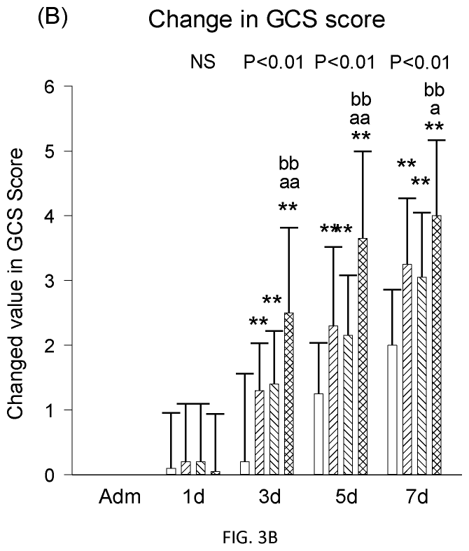

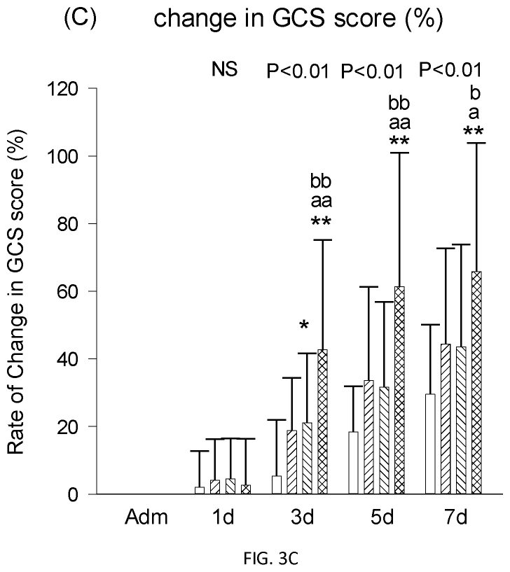

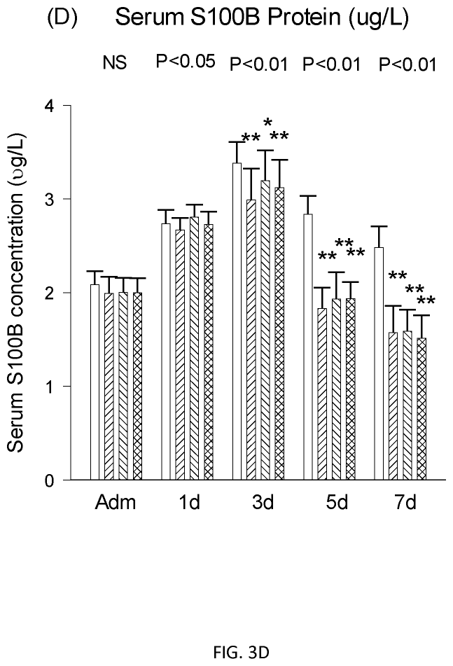

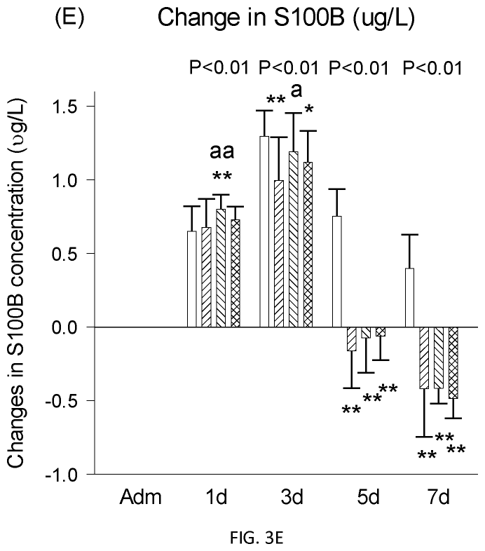

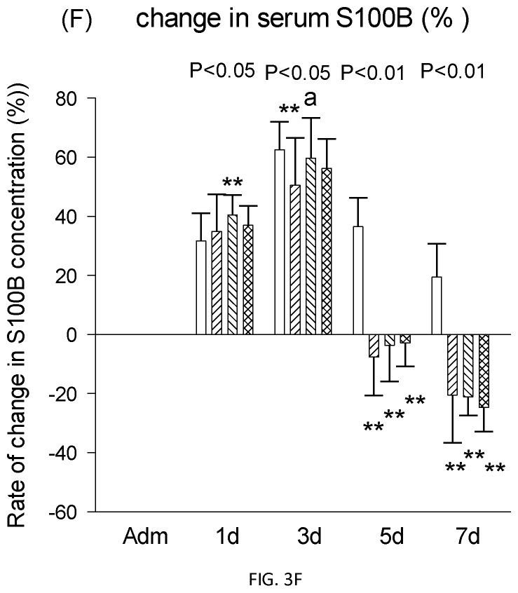

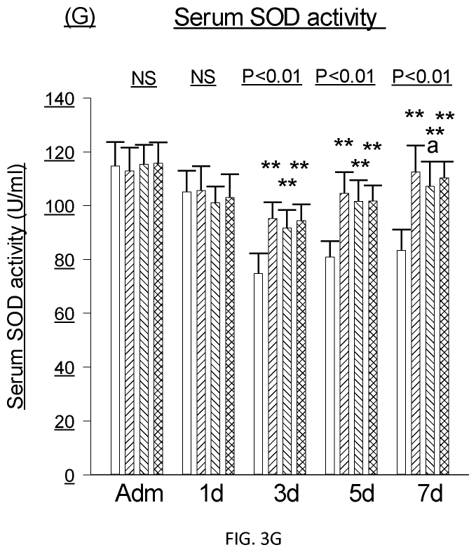

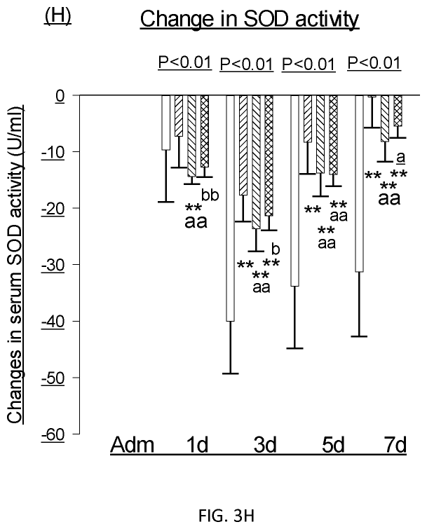

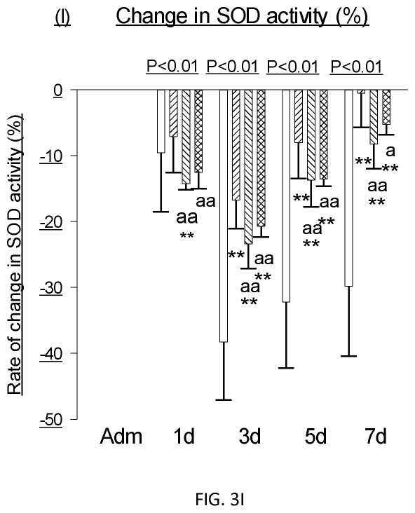

The present disclosure involves the demonstration by repeated-measures analysis of variance (RM-ANOVA) and/or analysis of variance (ANOVA) of serum S100B concentration as an indicator of secondary brain injury that shows significantly better efficacy of YB and XNJ treatment than OT (P<0.001), time (P<0.001) and time x treatment interaction (P<0.001) on S100B. Serum S100B concentration increased rapidly after craniotomy in all groups and peaked on Day 3 before S100B fell more rapidly and became significantly lower (P<0.01) in the XNJ, 1-YB, and h-YB groups than in the OT group on Days 3, 5, and 7 (Tables 2 and 3, FIGS. 2 and 3). The rate of change (%) in serum S100B concentration was significantly greater (P<0.05) in the 1-YB group than in the OT group on Day 1 post-surgery, and significantly greater (P<0.01) in the OT group than in the XNJ and h-YB groups on Day 3 post-surgery, whereas the absolute reduction and percentage reduction in serum S100B level were significantly greater in the XNJ, 1-YB and h-YB groups than in the OT group since Day 5 post-surgery. On Day 7 post-surgery, S100B declined to 20.5%, 20.1%, and 24.7% below baselines in the XNJ, 1-YB, h-YB groups, respectively, whereas it remained 19.4% above baseline in the OT group.

The present disclosure involves the demonstration that YB and XNJ interventions resulted in significantly better acute recovery and long-term improvements in clinical prognosis and functional recovery. Further, the high dose of YB resulted in significant (rate) increases in GCS score than 1-YB and XNJ 3 days post-surgery, suggesting more effective intervention after high dose YB administration, whereas XNJ therapy resulted in more consistent and significantly less (rate of) reduction in SOD activity than YB, indicating a greater antioxidative potential of XNJ than YB. The relatively greater loss in SOD activity in the 1-YB group than in the h-YB group on Days 1 and 3 and a great rate of increase in S100B in 1-YB than in XNJ on Day 3, indicating a dose-dependent neuroprotection of YB, possibly related to the multi anti-hemorrhage, anti-hemostasis, and anti-inflammation properties of YB as discussed below.

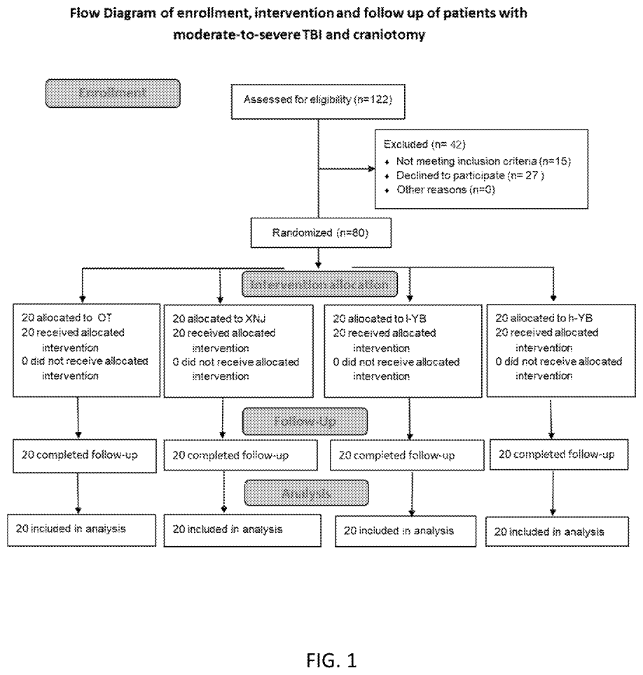

The present disclosure involves the demonstration of the use of a 4-arm parallel, randomized controlled trial design that involves 80 severity-matched TBI patients as depicted in the flow chart of enrollment, randomization, and experimental procedures (FIG. 1).

### Yunnan Baiyao (YB) Composition:

a. Yunnan Baiyao is a GMP-certified, standardized capsule formulation manufactured by Yunnan Baiyao Group Co., Ltd.
b. Each 500 mg unit contains: Panax notoginseng root (200 mg), Boea clarkeana (85 mg), Inula cappa (25 mg), Lycopodium complanatum (57.5 mg), Dioscorea opposita (66.5 mg), Alpinia officinarum (30 mg), and Erodium stephanianum (36 mg).
c. These ingredients are consistent with pharmacopeial disclosures, published clinical reports, and FDA-imported product labeling.

### Xingnaojing (XNJ) Composition:

a. Xingnaojing is an intravenous preparation manufactured by Wuxi Jimin Xinshanhe Pharmaceutical Co., Ltd.
b. Each 1,000 mL stock solution contains: Moschus (7.5 g), Radix Curcumae (30 g), Gardenia jasminoides (30 g), and Borneolum (1 g).
c. This formulation has been publicly disclosed in CN patent publications and peer-reviewed studies.

Regulatory-facing identity/status documentation for XNJ key constituents (supporting written description/enablement):
a. Gardeniae Fructus (Gardenia jasminoides) constituent genipin: FDA-listed color additive ‘Gardenia (genipin) blue’ (21 CFR 73.168) and related Federal Register actions (2025) (FDA press announcement (Gardenia (genipin) blue approval; Jul. 14, 2025); Federal Register order (Listing of Color Additives Exempt from Certification; Gardenia (Genipin) Blue; Jul. 15, 2025); FDA Color Additives database entry: Gardenia (Genipin) Blue (21 CFR 73.168); eCFR: 21 CFR 73.168—Gardenia (genipin) blue; Federal Register: Gardenia (Genipin) Blue; Confirmation of Effective Date (Oct. 1, 2025); FDA GRAS Notice Inventory (searchable; for GRAS correspondence on saffron extracts/constituents) FDA GInAS entry: CROCIN (ingredient record)).
b. Borneolum (borneol): FDA ‘Substances Added to Food’/EAFUS entry (BORNEOL; CAS 507-70-0) and CFR listing for synthetic flavoring substances (21 CFR 172.515). (FDA Substances Added to Food (formerly EAFUS) entry: BORNEOL (CAS 507-70-0); eCFR: 21 CFR 172.515-Synthetic flavoring substances and adjuvants; FDA Food Additive Status List/inventory portal (overview)}
c. Curcuma-derived constituents (curcumin/curcuminoids): FDA GRAS Notice dossiers (e.g., GRN 000686; GRN 000822) documenting identity and use in U.S. regulatory context GRAS Notice (GRN 000686): Curcumin from turmeric (Curcuma longa L.)-FDA GRAS Notice dossier (PDF); GRAS Notice (GRN 000822): Synthetic curcumin-FDA ‘no questions’ response letter (PDF).
d. Moschus/musk constituents (e.g., muscone): FDA ingredient records and import/identity tools. (FDA GInAS entry: MOSCHUS MOSCHIFERUS MUSK SAC RESIN (ingredient record); FDA Substances Added to Food (formerly EAFUS) entry: MUSK TONQUIN (MOSCHUS MOSCHIFERUS L.)
e. These FDA/US-government-facing sources are cited for identity/status and public availability of ingredient classes, not as assertions of FDA approval for the claimed TBI indication.

### Clinical Dosing Regimens:

a. YB was administered at either 1,000 mg/day (four 250 mg doses) or 2,000 mg/day (four 500 mg doses), given orally or via stomach tube, for seven consecutive days in an ICU setting.
b. XNJ was administered intravenously at 20 mL/day, diluted in 500 mL sterile saline, infused once daily for seven consecutive days.
c. These regimens were standardized, reproducible, and consistent with hospital practices.

### Clarification of Ingredient Certainty:

Stroke-rich evidence base and mechanistic rationale supporting XNJ use in acute brain injury (relevant to TBI secondary injury):
XNJ and its principal ingredient classes (Gardenia constituents such as geniposide/genipin and crocin/crocetin; Curcuma constituents; borneol; and muscone-related constituents) have extensive peer-reviewed preclinical and clinical literature in ischemic and hemorrhagic stroke, where hypoperfusion/hypoxia, oxidative stress, blood-brain barrier dysfunction, and neuroinflammation drive secondary injury mechanisms that overlap with post-TBI secondary injury cascades.
Song. Efficacy and safety of Xingnaojing injection for post-operative patients of intracerebral haemorrhage: a meta-analysis and systematic review. Front Pharmacol 2024; 15:1411026. doi: 10.3389/fphar.2024.1411026.
Wang. Efficacy and Safety of Xingnaojing Injection for Emergency Treatment of Acute Ischemic Stroke: A Systematic Review and Meta-Analysis. Front Pharmacol 2022; 13:839305. doi: 10.3389/fphar.2022.839305.
Tian. Chinese Herbal Medicine Xingnaojing Injection for Acute Ischemic Stroke: An Overview of Systematic Reviews and Meta-Analyses. Front Pharmacol 2021; 12:659408. doi: 10.3389/fphar.2021.659408.
Ma. Role of Xingnaojing Injection in treating acute cerebral hemorrhage: A systematic review and meta-analysis. Medicine (Baltimore) 2020; 99 (15): e19648. doi: 10.1097/MD.0000000000019648.
Yang. Chinese Herbal Medicine Xingnaojing Injection ( ) for Hypoxic Ischemic Encephalopathy in Newborns: A Systematic Review and Meta-Analysis. Chin J Integr Med 2018; 24 (2): 147-155. doi: 10.1007/s11655-015-1974-z.
Representative mechanistic examples include PI3K/Akt-mediated eNOS phosphorylation (Evid Based Complement Alternat Med. 2018; DOI: 10.1155/2018/2361046) and SIRT1-mediated suppression of inflammatory responses in ischemia/reperfusion models (Pharm Biol. 2020; 58 (1): 16-24; DOI: 10.1080/13880209.2019.1698619).

This stroke-rich evidence supports (i) identity/standardization and common dosing conventions for XNJ in acute brain injury care pathways, and (ii) mechanistic plausibility for mitigation of oxidative/inflammatory secondary injury following craniotomy-treated moderate-to-severee TBI, while the claimed clinical use remains specifically anchored to the disclosed TBI/craniotomy randomized trial outcomes.

XNJ has been widely reported and clinically used in hemorrhagic and ischemic stroke pathways. Thus, XNJ is traditionally viewed as a known agent in acute cerebrovascular care that is directed to stroke. However, the practice of the current disclosure has employed XNJ for a distinct and clinically constrained method-of-use—treating moderate-to-severee traumatic brain injury in patients undergoing emergency craniotomy—where the perioperative context, injury mechanism, and management goals differ materially from stroke care.

Non-obviousness wedge (stroke/TBI-with-craniotomy): Stroke publications do not teach or suggest the claimed peri-craniotomy TBI regimen (timing window, ICU course) or the prespecified endpoint package anchored to serum S100B and SOD trajectories with neurologic recovery outcomes (GCS/GOS/KPS). The disclosed use addresses secondary-injury biology predominant in post-craniotomy TBI (contusion/hemorrhage burden, reperfusion/edema dynamics, oxidative stress, and inflammatory cascades) and provides measurable intermediate endpoints (S100B reduction and SOD preservation/restoration) demonstrating benefit in a population and procedural context not claimed in the prior art.

Standardization and identity (non-limiting): In one embodiment, XNJ is identified and documented by product name, manufacturer/lot number, and a set of assayable marker constituents consistent with pharmacopeial or manufacturer specifications. Non-limiting examples of marker classes include geniposide-related iridoid glycosides (Gardenia), crocin/crocetin-related carotenoids, borneol-related monoterpenes, and Curcuma-related sesquiterpenes. Regulatory-facing ingredient identity/status documentation (e.g., U.S. FDA food additive/color additive/GRAS notices or ingredient safety dossiers may be used to support a clear identity and safety framing for these ingredient classes without limiting the claimed use to any single marker.: (FDA press announcement (Gardenia (genipin) blue approval; Jul. 14, 2025); Federal Register order (Listing of Color Additives Exempt from Certification; Gardenia (Genipin) Blue; Jul. 15, 2025); FDA Color Additives database entry: Gardenia (Genipin) Blue (21 CFR 73.168); eCFR: 21 CFR 73.168-Gardenia (genipin) blue; Federal Register: Gardenia (Genipin) Blue; Confirmation of Effective Date (Oct. 1, 2025); FDA GRAS Notice Inventory (searchable; for GRAS correspondence on saffron extracts/constituents); FDA GInAS entry: CROCIN (ingredient record); GRAS Notice (GRN 000686): Curcumin from turmeric (Curcuma longa L.)-FDA GRAS Notice dossier (PDF); GRAS Notice (GRN 000822): Synthetic curcumin-FDA ‘no questions’ response letter (PDF); FDA GInAS entry: MOSCHUS MOSCHIFERUS MUSK SAC RESIN (ingredient record); FDA Substances Added to Food (formerly EAFUS) entry: MUSK TONQUIN (MOSCHUS MOSCHIFERUS L.)

In one embodiment, the administered XNJ product is manufactured under quality-controlled conditions (e.g., GMP or equivalent) with batch release specifications suitable for clinical use and documentation in the medical record. Minor excipient variation, where present, is within acceptable pharmaceutical excipient tolerances and does not alter the claimed method-of-use.
a. In one embodiment, the administered XNJ product is manufactured under quality-controlled conditions (e.g., GMP or equivalent) with batch release specifications suitable for clinical use and documentation in the medical record. Minor excipient variation, where present, is within acceptable pharmaceutical excipient tolerances and does not alter the claimed method-of-use.
b. Minor excipient variation is clinically irrelevant and within FDA/CFDA excipient policy tolerances.

### Observed Clinical Effects:

a. The administration of both products is supported by randomized controlled trial (RCT) evidence in patients undergoing emergency craniotomy for moderate-to-severee TBI.
b. Outcomes include:
   Improvement in acute neurological recovery (GCS score at Days 1-7),
   Improved long-term outcomes (GOS and KPS at 30 and 90 days),
   Biomarker modulation (reduced serum S100B and preserved/restored SOD activity).
c. These results were reproducible and statistically significant compared with orthodox therapy (OT) alone.

The practice of the current disclosure has employed, unless otherwise indicated, conventional clinical methodology of orthodox therapies, traditional medicine and ethnic medicine in the management of patients with acute TBI and craniotomy within the skill of the art. Such techniques are explained fully in the literature. (e.g.: The Guideline for the management of patients with severe TBI (Neurosurgery. 2017 Jan. 1; 80 (1): 6-15),
Dai, C., Liang, Y., Hao, H., Z Heng, X., Xie, L., Guan, T., Zhou, Y., Wang, G., 2013. Global detection and identification of components from Yunnan Bai yao based onliquid chromatography hybridiontraptime-of-flight mass spectrometry. J. Sep. Sci. 36,1935-1944.
Einav, S., Y. Shoshan, H. Ovadia, I. Matot, M. Hersch, and E. Itshayek. 2006. Early postoperative serum S100 beta levels predict ongoing brain damage after meningioma surgery: a prospective observational study. Crit Care 10 (5): R141.
Glass, N. E., A. Vadlamani, F. Hwang, Z. C. Sifri, A. Kunac, S. Bonne, S. R. Pentakota, P. Yonclas, A. C. Mosenthal, D. H. Livingston, and J. S. Albrecht. 2019. Bleeding and Thromboembolism After Traumatic Brain Injury in the Elderly: A Real Conundrum. J Surg Res 235:615-620.
Ladas, E. J., Karlik, J. B., Rooney, D., Taromina, K., Ndao, D. H., Granowetter, L., Kelly, K. M., 20 12. Topical YunnanBaiyaoadministrationasanadjunctivetherapyforbleedingcomplicationsin adolescentswithadvancedcancer.Support.CareCancer20,3379-3383;
Tang, Z. L., X. Wang, B. Yi, Z. L. Li, C. Liang, and X. X. Wang. 2009. Effects of the preoperative administration of Yunnan Baiyao capsules on intraoperative blood loss in bimaxillary orthognathic surgery: a prospective, randomized, double-blind, placebo-controlled study. Int J Oral Maxillofac Surg 38 (3): 261-266;
Wang, J. C., Q. H. Wang, and J. X. Tian. 2015. Therapeutic effect of integrated traditional Chinese and Western medicine on patients with mental disorders caused by traumatic brain injury. Chin. J. Pract. Nerv. Dis. 18 (8): 50-52;
Xu, M., W. Su, Q. P. Xu, and W. D. Huang. 2010. Effect of Xingnaojing injection on cerebral edema and blood-brain barrier in rats following traumatic brain injury. Chin J Traumatol 13 (3): 158-162;
Xu, D., P. Huang, Z. Yu, D. H. Xing, S. Ouyang, and G. Xing. 2014. Efficacy and Safety of Panax notoginseng Saponin Therapy for Acute Intracerebral Hemorrhage, Meta-Analysis, and Mini Review of Potential Mechanisms of Action. Front Neurol 5:274; Xu, Y. M., X. C. Wang, S. J. Zhang, T. T. Xu, H. Y. Li, S. Y. Hei, Z. H. Wen, Y. Z. Ma, Q.
Wang, and W. X. Liang. 2018. Role of Xingnaojing combined with naloxone in treating intracerebral haemorrhage: A systematic review and meta-analysis of randomized controlled trials. Medicine (Baltimore) 97 (43): e12967.

At admission, all TBI patients underwent a complete medical history inquiry, physical examination, routine laboratory tests, and a cranial CT scan. The demographic and general clinical diagnosis at admission includes scoring, degree of Glasgow Coma Scale (GCS), type and cause of injury, and type of craniotomy. Emergency craniotomy included intracranial hematoma evacuation, inactivated brain tissue resection, bone flap decompression, and so on.

The inclusion criteria of the participants include:
1) diagnosed with moderate-to-severee TBI (within 12 hours of TBI, Glasgow Coma Scale score, 3~12), without apparent injury to other organs;
2) intracranial contusion/laceration, intracranial/epidural/subdural hematoma, and other organic lesions confirmed by computed tomography (CT);
3) between 15 and 65 years of age; 4) received emergency craniotomy within 12 hours of TBI.
4) The patients or all their legal guardians gave their informed written consent.

The exclusion criteria of the participants include:
1) severe multiple or combined injuries;
2) history of severe chronic illness;
3) participated in any drug trial one month prior to this trial;
4) had a malignant tumor or other diseases in the nervous or immune systems;
5) pregnant and lactating women;
6) with more than 400 ml of blood transfusion during treatment;
7) uncooperative;
8) automatic discharge or death during the assessing phase;
9) had a second craniotomy;
10) history of allergy to YB or intravenous XNJ.

All study procedures were conducted in accordance with the Helsinki Declaration of 1975, in adherence to CONSORT guidelines (consort-statement.org/), and were approved by the Ethics Committee of the conducting hospital. The clinical trial was registered at Chinese Clinical Trial Registry (ChiCTR2000030280 and ChiMCTR2000003057).

### Randomization

After craniotomy, TBI patients (N=80) were randomly assigned to one (n=20) of the four following treatments for 7 days by the clinicians using a predetermined randomization code generated by a random number generator (G.B.) according to the time order of admission: 1) orthodox treatment (OT); 2) OT plus Xingnaojing (XNJ) (intravenous drip of 20 ml Xingnaojing in 500 ml normal saline, daily, Wuxi Jimin Xinshanhe Pharmaceutical Co., Ltd., China); 3) OT plus low dose Yunnan Baiyao (I-YB) (1,000 mg, q.i.d) (Yunnan Baiyao Group Co., Ltd., China, oral or via stomach tube, q.i.d.); 4) OT plus high dose YB (h-YB) (2,000 mg, q.i.d). Orthodox treatments (OT) include dehydration, hemostasis, diuresis, prophylactic dose of antibiotics, hormone therapy, prevention of epilepsy and other necessary symptomatic treatments. Medical treatment was administered daily by caring nurses. Changes in clinical symptoms, including vital signs, conscious state, pupil changes, etc. were scored daily.

The present disclosure involves the primary outcome measures of the therapeutic effects of OT, YB, and XNJ of TBI and craniotomy, including measures of changes in postoperative scores of Glasgow Coma Scale (GCS), post-discharge scores of Glasgow Outcome Scale (GOS) and Karnofsky Performance Scale (KPS) evaluated by trained neurologists. The GCS was used to evaluate disturbance of consciousness with eye opening, speech condition, and motor response (mild=13-15, moderate=8-12, severe=3-7). The outcome of GOS was classified as good (4-5), bad (2-3), and death (1). KPS reflects changes in physical strength, functional rehabilitation, and the abilities of daily life and work. A score of >80-points indicates independent, adequate capability of self-care in daily life; a score of 60~70 points indicates semi-dependent in daily life, and a score of <60-points indicates a lack of self-care capability.

The present disclosure involves the measures of the secondary brain injury biomarkers of the TBI patients, including the changes in serum S100B protein and serum superoxide dismutase (SOD) activity. Five ml of venous blood was drawn on admission and on postoperative Days 1, 3, 5, and 7. After 2000 r/min centrifugation, the serum was stored at −80° C., and thawed at 4° C. overnight before assaying. Serum S100B was determined by an enzyme-linked immunosorbent assay (Elisa) kit for human serum S100B protein (Randy D Company, USA), and serum superoxide dismutase (SOD) activity was determined by the pyrogallol autoxidation method (PAM) using the SOD detection kit (Fujian Fuyuan Biotechnology Co., Ltd., China).

Methods of OT, YB, and XNJ Administration and Delivery Forms: The present disclosure involves the oral administration or alternative stomach tubing administration of YB capsule (250 mg/each, a proprietary product of Yunnan Baiyao Group Co., Ltd. Yunnan, China). The present disclosure involves the intravenous drip administration of Xingnaojing (XNJ) (20 ml XNJ in 500 ml normal saline, XNJ is a trademark product of Wuxi Jimin Xinshanhe Pharmaceutical Co., Ltd., Wuxi, China).

The present disclosure involves orthodox treatments (OT) of TBI patients, including dehydration, hemostasis, diuresis, prophylactic dose of antibiotics, hormone therapy, prevention of epilepsy and other necessary symptomatic treatments. Medical treatment was administered daily by hospital nurses. Changes in clinical symptoms, including vital signs, conscious state, pupil changes, etc. are scored daily by trained neurologists.

### Dosages of YB and XNJ Administration:

Low dose of Yunnan Baiyao (1-YB), 1,000 mg/day, q.i.d, (oral or via stomach tube) High dose of Yunnan Baiyao (h-YB), 2,000 mg/day, q.i.d, (oral or via stomach tube) Xingnaojing (XNJ) 20 ml Xingnaojing in 500 ml normal saline (intravenous drip)

Participant sample size of this disclosure was estimated to be 20 participants per group or a total of 80 participants for the 4-arm trial when GCS scores were chosen as the main outcomes, with a designed power of 0.9, and assuming no baseline difference in GCS scores (8.0 for all groups) and assuming that after 7-days of OT and OT+YBY/XNJ interventions it would result in a 2-unit difference in mean GCS score between OT (10.0) and each of other 3 groups (12.0) with a standard deviation of 1.6. Similarly, if assuming 20 differences in mean GOS score outcome and 15 in standard deviation between the OT group (40.0) and the treatment groups (60.0) after 30 days of emergency craniotomy discharge, the minimal sample size would be 17 participants per treatment group or a total of 68 participants.

Normally distribution of GCS, S100B, SOD, GOS, and KPS was confirmed, and the data of GCS, S100B, SOD, GOS, and KPS were presented. General linear model of repeated measures was performed to measure the effects of treatments, time and treatment*time interactions. Changes and change rate (%) of the measured variables at each postoperative observation time over the baseline (on admission) were analyzed using independent t-tests, ANOVA, or nonparametric Mann-Whitney U-test as appropriate (SPSS 24, USA). Categorical variables were tested with a χ²-test. Pearson correlations between the variables were calculated. A two-tailed P value<0.05 was considered statistically significant.

Traffic accident was the main cause of TBI, and brain contusion was the primary damage type in this study (Table 1). There were no baseline differences in demographic and clinical characteristics, including age, gender, GCS score, type and cause of TBI, and type of craniotomy, between the four treatment groups: OT, XNJ+OT, 1-YB+OT, and h-YB+OT.

Repeated measure analysis showed significant effects of treatment (P<0.001), time (P<0.001) and time x treatment interaction (P<0.001) and a trend effect of treatment (P=0.2) on GCS scores observed during the first 7-day period post craniotomy that were confirmed by ANOVA (Table 2, FIGS. 2 and 3A-3C). GCS score was lowest at admission in all groups but improved more quickly and became significantly greater in the XNJ, 1-YB, h-YB groups than in the OT group since Day 3 (P<0.01). The improvement rate in GCS were significantly greater in h-YB than in the 1-YB and XNJ groups on Days 3, 5, and 7 (P<0.01, all) which may also reflect a relatively lower baseline GCS value in the h-YB group (7.30±2.43) than in the OT, XNJ, and 1-YB groups (8.10±2.38, 8.60±2.50, 8.50±2.48, respectively).

Repeated measure analysis and ANOVA analysis showed significant effects of treatment (P0.05) (Table 2, FIG. 2). One month after craniotomy the scores of GOS (3.80±0.95, 3.70±0.57, 3.70±0.87) and KPS (62.50±20.49, 59.50±10.99, 58.50±17.25) in the XNJ, 1-YB, h-YB groups were 27.5%~31% and 50%~60% significantly greater than that in OT group (2.90-1.07 and 39.00±21.25) (P<0.01, all), respectively. Similar differences remained 3 months after surgery.

Repeated serum S100B and SOD measure analysis and ANOVA showed significant effects of Treatment (P<0.001), Time (P<0.001) and Time x Treatment interaction (P<0.001) on serum S100B concentration that increased rapidly after surgery and peaked in all groups on Days 3 before fell more rapidly and became significantly lower (P<0.01) in the XNJ, 1-YB and h-YB groups than in the OT group on Days 3, 5, and 7 (Table 2, FIGS. 2B and 3D-3F). The rate of change (%) in serum S100B was significantly greater (P<0.05) in the 1-YB than in the OT group on Day 1 of the treatment, and significantly greater (P<0.01) in the OT group than in the XNJ and h-YB groups on Day 3 of the treatment whereas reduction and percentage reduction in serum S100B level was significantly greater in the XNJ, 1-YB and h-YB groups than in the OT group since Day 5 of treatment. On Day 7, S100B declined to 20.5%, 20.1%, and 24.7% below baselines in the XNJ, 1-YB, and h-YB groups, respectively, whereas it remained 19.4% above baseline in OT group.

Prediction of long-term TBI outcomes based on correlations between experimental variables: GCS score was significantly correlated with GOS/KPS scores in pooled TBI patients and in each of the treatment groups (P<0.01) (Table 3), indicating a predictive value of postoperative recovery during the acute phase of post-surgery on long-term clinical prognosis of TBI. It is noticed that the correlation between GOS/KPS and GCS scores improved progressively from Day 1 to Day 7 after surgery. However, the reduced level of correlation between most variables immediately after the surgery (Day 1) indicates a disruptive effect of craniotomy. Serum S100B levels were negatively correlated with GCS/GOS/KPS scores in pooled patients and in each treatment group (Table 3). This and other analyses suggest that a higher S100B level on admission day or on Day 7 are better predictor of poor TBI outcomes (low GCS/GOS/KPS scores). In contrast, serum SOD activity was positively correlated with GCS/GOS/KPS scores (P<0.01) in the pooled and in the 3 co-treatment groups but not in the OT group. Similarly, SOD level on postoperative Day 7 is a better predictor of GCS/GOS/KPS scores than SOD on other days in the pooled and 3 adjuvant treatment groups, but not the OT group. Our data suggest that GCS and SOD are positive predictors, and S100B is a negative predictor of clinical prognosis.

### XNY: Clinical Endpoint and Biomarker Coupling (Non-Hemodynamic)

The administration of XNJ is not asserted to require or produce any vascular-perfusion or hemodynamic-rescue effect as a claim limitation. Instead, the administration of XNJ is directed to attenuation of secondary-injury burden as reflected by (i) biomarker modulation limited to serum S100B and SOD activity, and (ii) functional recovery endpoints (GCS, GOS, and KPS) in a clinically defined, peri-craniotomy moderate-to-severee TBI population.

In one embodiment, traumatic brain injury requiring emergency craniotomy represents a high-risk clinical setting characterized by substantial secondary injury evolution after surgical decompression and hematoma evacuation. In this setting, clinically meaningful benefit is demonstrated by improved neurological recovery during acute hospitalization and improved longer-term functional prognosis, supported by mechanistically coherent intermediate endpoints.

One technical benefit of an embodiment is to limit the clinical context and tether the administration of XNJ to a measurable endpoint signature. The signature includes (a) improved acute consciousness recovery as measured by serial GCS assessment; (b) improved post-discharge prognosis as measured by GOS and KPS; and (c) favorable trajectories of serum S100B and serum SOD activity sampled at prespecified timepoints.

In one embodiment, a randomized controlled trial regimen is configured to enable XNJ administration in the peri-craniotomy acute phase. In one clinically supported embodiment, XNJ is administered as adjunct therapy to orthodox postoperative management in moderate-to-severee TBI patients who undergo emergency craniotomy within 12 hours of injury. In an embodiment, after surgery, the patients are assigned to receive orthodox therapy alone or orthodox therapy plus XNJ for seven consecutive days. XNJ dosing and administration format is: XNJ is delivered by intravenous drip as 20 mL XNJ diluted in 500 mL normal saline, administered once daily for 7 days.

In one embodiment, orthodox therapy includes clinically indicated postoperative management such as dehydration therapy, hemostasis measures, diuresis, prophylactic antibiotics, hormone therapy, seizure prevention, and other symptomatic treatments, with daily documentation of vital signs and neurological status. This dosing and timing framework provides explicit enablement of XNJ use in the intensive-care perioperative setting, including controllable parenteral administration compatible with standard nursing workflows.

In one embodiment, the use of XNJ includes a sampling schedule coupled to a plurality of prespecified endpoints by biomarker-outcome coupling. In this clinical-use context, disturbance of consciousness is evaluated using GCS during the seven-day postoperative recovery period. Post-discharge prognosis is evaluated by GOS and KPS at 30 and 90 days after surgery.

In one embodiment, GOS categories include good outcome (scores 4-5), bad outcome (scores 2-3), and death (score 1). KPS is interpreted as follows: scores greater than 80 reflect independence and adequate self-care; scores of 60-70 reflect semi-dependence; and scores less than 60 reflect lack of self-care capability.

In one embodiment, the use of XNJ includes a plurality of biomarker endpoints. Venous blood is sampled at admission (baseline) and on postoperative Days 1, 3, 5, and 7. Serum is prepared by centrifugation and stored under conditions suitable for later quantification of serum S100B concentration and serum SOD activity by validated laboratory assays. Thus, the XNJ use provides a functional and enabled application by coupling functional endpoints to biomarker trajectories. Specifically, the present disclosure contemplates the reduction of serum S100B and preservation or recovery of serum SOD activity over the acute postoperative course co-occur with improved GCS recovery and improved GOS/KPS prognosis. This coupling framework defines intermediate endpoints and prespecified timepoints that are clinically meaningful, measurable, and linked to functional recovery in a defined TBI craniotomy population.

In one embodiment, secondary injury after severe TBI and craniotomy is associated with oxidative stress amplification that can worsen cellular injury cascades. Accordingly, XNJ attenuates the oxidative-stress burden during acute postoperative recovery as reflected by preservation, reduced loss, or faster recovery of serum SOD activity relative to control.

In one embodiment consistent with the clinical evidence, serum SOD activity is assessed at admission and on Days 1, 3, 5, and 7, and XNJ administration is associated with a smaller early postoperative decline and/or a larger recovery fraction by Day 7 compared with orthodox therapy alone. However, prior experience that describes XNJ in broad neurologic or emergency settings does not teach or enable this biomarker-tethered use in moderate-to-severee TBI patients undergoing emergency craniotomy, including the prespecified SOD sampling schedule coupled to functional endpoints.

### XNJ and Injury-Biomarker Attenuation (S100B-Anchored Embodiment)

Non-limiting biomarker-guided administration example: In one embodiment, clinicians use serial S100B and SOD results to guide continuation or extension of adjunctive XNJ dosing during the first 7 postoperative days. For example, if S100B fails to decline relative to a patient's prior measurement and/or remains above an institution-defined high-risk threshold, and/or if SOD activity shows a persistent decline without recovery, XNJ dosing may be continued through Day 7; whereas if S100B shows a consistent downward trajectory with stabilization and SOD shows recovery toward baseline, dosing may be discontinued earlier. These decision criteria may be implemented using absolute values or percent change (e.g., day-to-day percent change) and are provided as examples to demonstrate enablement and clinical operability.

In one embodiment, for patients administered with XNJ, serum S100B is used as a clinically informative biomarker associated with brain injury burden and postoperative course. As a result, XNJ reduces the magnitude and/or duration of postoperative S100B elevation and accelerates return toward baseline in the acute recovery window.

In one embodiment consistent with the clinical evidence, serum S100B is quantified at admission and on Days 1, 3, 5, and 7. XNJ administration is associated with a lower S100B trajectory at later postoperative timepoints compared with orthodox therapy alone.

This XNJ administration associated with the S100B-anchored biomarker is used to provide measurable intermediate endpoints and sampling time points that were not used in traditional methods applied to this particular peri-craniotomy TBI population.

In one embodiment, XNJ improves the rate of neurological recovery during acute hospitalization as assessed by serial GCS scoring, and improves clinical prognosis as assessed by GOS and KPS at post-discharge follow-up.

In one embodiment, GCS is measured daily during the seven-day postoperative intervention period, and XNJ administration is associated with earlier or greater improvement in GCS compared with orthodox therapy alone.

In one embodiment, GOS and KPS are assessed at 30 and 90 days after surgery, and XNJ administration is associated with better prognosis and functional status compared with orthodox therapy alone.

In one embodiment, these functional endpoints correlate with biomarker trajectories (S100B and SOD) in order to support a coherent endpoint package that is clinically meaningful and measurable in the acute peri-craniotomy context.

In one embodiment, the disclosed use of XNJ for emergency craniotomy-associated secondary injury is distinct from prior XNJ uses. In particular, the disclosed use of XNJ is clinically separated from generic disclosures of XNJ by the treatment context and endpoint package. Emergency craniotomy patients represent a distinct population with iatrogenic surgical stress and evolving postoperative secondary injury. The disclosed use of XNJ therefore limits XNJ use to defined perioperative timing, parenteral dosing format, and prespecified functional and biomarker endpoints.

Accordingly, even where XNJ is known as a product, prior art does not disclose or enable the present method-of-use in moderate-to-severee TBI patients undergoing emergency craniotomy within 12 hours of injury, administered as 20 mL in 500 mL normal saline daily for 7 days, with biomarker sampling at admission and Days 1, 3, 5, and 7 and functional follow-up at 30 and 90 days.

In summary, the disclosed use of XNJ is directed to attenuation of secondary-injury burden by (i) anchoring XNJ to a defined peri-craniotomy moderate-to-severee TBI population, (ii) specifying a concrete IV dosing regimen and sampling schedule, and (iii) coupling biomarkers limited to S100B and SOD to functional endpoints (GCS, GOS, KPS) to support novelty, non-obviousness, and multiple fallback claim positions. The disclosed use of XNJ does not require demonstrating cerebral blood-flow augmentation or hemodynamic rescue as a claim limitation.

In one embodiment, the disclosed use of XNJ acts as an independent adjunctive agent for TBI, clinically constrained to a peri-craniotomy ICU population and tethered to prespecified functional endpoints (GCS/GOS/KPS) and biomarker trajectories (S100B/SOD), without asserting hemodynamic rescue for XNJ. This structure is intended to (i) address any prior art/general-use concerns by narrowing to a defined clinical context and endpoint package, and (ii) strengthen written description/enablement by specifying identity/composition and a concrete IV regimen (dose, dilution, administration format, duration).

In one embodiment, the disclosed use of XNJ is capable of treating moderate-to-severee traumatic brain injury in a human patient undergoing emergency craniotomy. The disclosed use of XNJ includes intravenously administering XNJ as adjunct therapy during an acute postoperative phase. The emergency craniotomy is performed within 12 hours of injury. XNJ is administered once daily for seven days. For example, XNJ is administered by intravenous drip as 20 mL XNJ diluted in 500 mL normal saline.

In one embodiment, the disclosed use of XNJ is capable of improving neurological recovery after emergency craniotomy in a moderate-to-severee TBI patient. The disclosed use of XNJ includes administering XNJ in an amount effective to improve the GCS score during a 7-day postoperative recovery period.

In one embodiment, the disclosed use of XNJ is capable of improving clinical prognosis after emergency craniotomy in a moderate-to-severee TBI patient, comprising administering XNJ in an amount effective to improve GOS and/or KPS at 30 and/or 90 days after surgery.

In one embodiment, the disclosed use of XNJ is capable of modulating secondary-injury biomarkers after emergency craniotomy in a moderate-to-severee TBI patient. The disclosed use of XNJ includes administering XNJ in an amount effective to (i) reduce serum S100B and/or (ii) preserve or increase serum superoxide dismutase (SOD) activity relative to control. Venous blood is sampled at admission and on postoperative Days 1, 3, 5, and 7 to quantify serum S100B and serum SOD activity. Reduction of serum S100B and preservation or recovery of serum SOD activity is coupled with improvement in GCS during hospitalization and improvement in GOS and/or KPS at follow-up.

The present disclosure demonstrates that Yunnan Baiyao (YB) and Xingnaojing (XNJ) were more effective than orthodox therapies for patients with mild-to-moderate TBI and craniotomy. The present disclosure addresses the urgent need for innovative therapies for acute TBI and craniotomy, especially in emergency situations and situations of limited resources. In the present disclosure, both YB and XNJ treatments resulted in similar significant improvements in neuroprotection and clinical prognosis. However, some differences do exist. The h-YB resulted in significant (rate of) increases in GCS score than 1-YB and XNJ three days after surgery, suggesting more effective intervention after high dose YB administration, whereas XNJ therapy resulted in more consistent and significantly less (rate of) reduction in SOD activity than YB, indicating a greater antioxidative potential of XNJ than YB. The greater loss in SOD activity in the 1-YB group than in the h-YB group on Days 1 and 3, and a great rate of increase in S100B in 1-YB than in XNJ on Day 3, indicating a dose-dependent neuroprotection of YB, possibly related to the multi anti-hemorrhage, anti-hemostasis, and anti-inflammation properties of YB.

The present disclosure methods pertain to the development of novel therapies for treatment and prevention of coagulopathy and secondary neural injury-associated neurodegeneration and neuroinflammation diseases, including TBI, spinal cord injury, stroke, brain tumor, ischemic and hemorrhage stroke that may involve neurosurgery or craniotomy.

In the foregoing specification, embodiments of the invention have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction.

All references cited in the present disclosure are incorporated by reference in their entirety. For an overall review of prior art solutions for TBI patients, the reader is directed to the following academic and patent publications.

### REFERENCES

Anderson, R. E., L. O. Hansson, O. Nilsson, R. Dijlai-Merzoug, and G. Settergren. “High Serum S100b Levels for Trauma Patients without Head Injuries.” Neurosurgery 48, no. 6 (June 2001): 1255-8; discussion 58-60. dx.doi.org/10.1097/00006123-200106000-00012.
Arcaroli, J. J., J. E. Hokanson, E. Abraham, M. Geraci, J. R. Murphy, R. P. Bowler, C. A. Dinarello, L. Silveira, J. Sankoff, D. Heyland, P. Wischmeyer, and J. D. Crapo. “Extracellular Superoxide Dismutase Haplotypes Are Associated with Acute Lung Injury and Mortality.” Am J Respir Crit Care Med 179, no. 2 (Jan. 15 2009): 105-12. dx.doi.org/10.1164/rccm.200710-15660C.
Berger, R. P., M. C. Pierce, S. R. Wisniewski, P. D. Adelson, R. S. Clark, R. A. Ruppel, and P. M. Kochanek. “Neuron-Specific Enolase and S100b in Cerebrospinal Fluid after Severe Traumatic Brain Injury in Infants and Children.” Pediatrics 109, no. 2 (February 2002): E31. dx.doi.org/10.1542/peds. 109.2.e31.
Berger, R. P., M. C. Pierce, S. R. Wisniewski, P. D. Adelson, and P. M. Kochanek. “Serum S100b Concentrations Are Increased after Closed Head Injury in Children: A Preliminary Study.” J Neurotrauma 19, no. 11 (November 2002): 1405-9. dx.doi.org/10.1089/089771502320914633.
Bokesch, P. M., E. Appachi, M. Cavaglia, E. Mossad, and R. B. Mee. “A Glial-Derived Protein, S100b, in Neonates and Infants with Congenital Heart Disease: Evidence for Preexisting Neurologic Injury.” Anesth Analg 95, no. 4 (October 2002): 889-92, table of contents. dx.doi.org/10.1097/00000539-200210000-00018.
Chen, L., H. Jiang, G. Xing, B. Guan, Y. Yang, A. Ahmed, and X. Ma “Effects of Yunnan Baiyao Adjunct Therapy on Postoperative Recovery And Clinical Prognosis of Patients with Traumatic Brain Injury: A Randomized Controlled Trial.” Phytomedicine, 89. (August 2021): 153593. dx.doi.org/10.1016/j.phymed.2021.153593.
Cheng, Y., J. Zan, Y. Song, G. Yang, H. Shang, and W. Zhao. “Evaluation of Intestinal Injury, Inflammatory Response and Oxidative Stress Following Intracerebral Hemorrhage in Mice.” Int J Mol Med 42, no. 4 (October 2018): 2120-28. dx.doi.org/10.3892/ijmm.2018.3755.
Clark, R. S., H. Bayir, C. T. Chu, S. M. Alber, P. M. Kochanek, and S. C. Watkins. “Autophagy Is Increased in Mice after Traumatic Brain Injury and Is Detectable in Human Brain after Trauma and Critical Illness.” Autophagy 4, no. 1 (January 2008): 88-90. ncbi.nlm.nih.gov/pubmed/17957135.
Clark, R. S., J. Chen, S. C. Watkins, P. M. Kochanek, M. Chen, R. A. Stetler, J. E. Loeffert, and S. H. Graham. “Apoptosis-Suppressor Gene Bcl-2 Expression after Traumatic Brain Injury in Rats.” J Neurosci 17, no. 23 (Dec. 1 1997): 9172-82. ncbi.nlm.nih.gov/pubmed/9364064.
Clark, R. S., P. M. Kochanek, P. D. Adelson, M. J. Bell, J. A. Carcillo, M. Chen, S. R. Wisniewski, K. Janesko, M. J. Whalen, and S. H. Graham. “Increases in Bcl-2 Protein in Cerebrospinal Fluid and Evidence for Programmed Cell Death in Infants and Children after Severe Traumatic Brain Injury.” J Pediatr 137, no. 2 (August 2000): 197-204. dx.doi.org/10.1067/mpd.2000.106903.
Clark, R. S., P. M. Kochanek, C. E. Dixon, M. Chen, D. W. Marion, S. Heineman, S. T. DeKosky, and S. H. Graham. “Early Neuropathologic Effects of Mild or Moderate Hypoxemia after Controlled Cortical Impact Injury in Rats.” J Neurotrauma 14, no. 4 (April 1997): 179-89. dx.doi.org/10.1089/neu. 1997.14.179.
Cox, A. L., A. J. Coles, J. Nortje, P. G. Bradley, D. A. Chatfield, S. J. Thompson, and D. K. Menon. “An Investigation of Auto-Reactivity after Head Injury.” J Neuroimmunol 174, no. 1-2 (May 2006): 180-6. dx.doi.org/10.1016/j.jneuroim.2006.01.007.
Einav, S., Y. Shoshan, H. Ovadia, I. Matot, M. Hersch, and E. Itshayek. “Early Postoperative Serum S100 Beta Levels Predict Ongoing Brain Damage after Meningioma Surgery: A Prospective Observational Study.” Crit Care 10, no. 5 (2006): R141. dx.doi.org/10.1186/cc5058.
Forbes, M. L., R. S. Clark, C. E. Dixon, S. H. Graham, D. W. Marion, S. T. DeKosky, J. K. Schiding, and P. M. Kochanek. “Augmented Neuronal Death in Ca3 Hippocampus Following Hyperventilation Early after Controlled Cortical Impact.” J Neurosurg 88, no. 3 (March 1998): 549-56. dx.doi.org/10.3171/jns.1998.88.3.0549.
Hutchinson, P. J., M. T. O'Connell, N. J. Rothwell, S. J. Hopkins, J. Nortje, K. L. Carpenter, I. Timofeev, P. G. Al-Rawi, D. K. Menon, and J. D. Pickard. “Inflammation in Human Brain Injury: Intracerebral Concentrations of Il-1alpha, Il-1beta, and Their Endogenous Inhibitor Il-1ra.” J Neurotrauma 24, no. 10 (October 2007): 1545-57. dx.doi.org/10.1089/neu.2007.0295.
Juul, K., A. Tybjaerg-Hansen, S. Marklund, N. H. Heegaard, R. Steffensen, H. Sillesen, G. Jensen, and B. G. Nordestgaard. “Genetically Reduced Antioxidative Protection and Increased Ischemic Heart Disease Risk: The Copenhagen City Heart Study.” Circulation 109, no. 1 (Jan. 6 2004): 59-65. dx.doi.org/10.1161/01.CIR.0000105720.28086.6C.
Kim, G. W., T. Kondo, N. Noshita, and P. H. Chan. “Manganese Superoxide Dismutase Deficiency Exacerbates Cerebral Infarction after Focal Cerebral Ischemia/Reperfusion in Mice: Implications for the Production and Role of Superoxide Radicals.” Stroke 33, no. 3 (March 2002): 809-15. dx.doi.org/10.1161/hs0302.103745.
Knoblach, S. M., M. Nikolaeva, X. Huang, L. Fan, S. Krajewski, J. C. Reed, and A. I. Faden. “Multiple Caspases Are Activated after Traumatic Brain Injury: Evidence for Involvement in Functional Outcome.” J Neurotrauma 19, no. 10 (October 2002): 1155-70. dx.doi.org/10.1089/08977150260337967.
Kobylecki, C. J., S. Afzal, and B. G. Nordestgaard. “Genetically Low Antioxidant Protection and Risk of Cardiovascular Disease and Heart Failure in Diabetic Subjects.” EBioMedicine 2, no. 12 (December 2015): 2010-5. dx.doi.org/10.1016/j.ebiom.2015.11.026.
Kontos, H. A. and E. P. Wei. “Superoxide Production in Experimental Brain Injury.” J Neurosurg 64, no. 5 (May 1986): 803-7. dx.doi.org/10.3171/jns. 1986.64.5.0803.
Lee, B., J. Leem, H. Kim, H. G. Jo, S. H. Yoon, A. Shin, J. U. Sul, Y. Y. Choi, Y. Yun, and C. Y. Kwon. “Herbal Medicine for Acute Management and Rehabilitation of Traumatic Brain Injury: A Protocol for a Systematic Review.” Medicine (Baltimore) 98, no. 3 (January 2019): e14145. dx.doi.org/10.1097/MD.0000000000014145.
Li, D. R., B. L. Zhu, T. Ishikawa, D. Zhao, T. Michiue, and H. Maeda. “Immunohistochemical Distribution of S-100 Protein in the Cerebral Cortex with Regard to the Cause of Death in Forensic Autopsy.” Leg Med (Tokyo) 8, no. 2 (March 2006): 78-85. dx.doi.org/10.1016/j.legalmed.2005.09.002.
Loane, D. J., B. A. Stoica, and A. I. Faden. “Neuroprotection for Traumatic Brain Injury.” Handb Clin Neurol 127 (2015): 343-66. dx.doi.org/10.1016/B978-O-444-52892-6.00022-2.
Lu, G., L. Zhu, X. Wang, H. Zhang, and Y. Li. “Decompressive Craniectomy for Patients with Traumatic Brain Injury: A Pooled Analysis of Randomized Controlled Trials.” World Neurosurg 133 (January 2020): e135-e48. dx.doi.org/10.1016/j.wneu.2019.08.184.
Muir, J. K., M. Tynan, R. Caldwell, and E. F. Ellis. “Superoxide Dismutase Improves Posttraumatic Cortical Blood Flow in Rats.” J Neurotrauma 12, no. 2 (April 1995): 179-88. dx.doi.org/10.1089/neu. 1995.12.179.
Muller, P., D. Jirsch, J. D'Sousa, C. Kerr, and C. Knapp. “Gastrointestinal Bleeding after Craniotomy: A Retrospective Review of 518 Patients.” Can J Neurol Sci 15, no. 4 (November 1988): 384-7. ncbi.nlm.nih.gov/pubmed/3208221.
Newcombe, V. F., G. B. Williams, J. Nortje, P. G. Bradley, D. A. Chatfield, J. G. Outtrim, S. G. Harding, J. P. Coles, B. Maiya, J. H. Gillard, P. J. Hutchinson, J. D. Pickard, T. A. Carpenter, and D. K. Menon. “Concordant Biology Underlies Discordant Imaging Findings: Diffusivity Behaves Differently in Grey and White Matter Post Acute Neurotrauma.” Acta Neurochir Suppl 102 (2008): 247-51. ncbi.nlm.nih.gov/pubmed/19388324.
Papa, L., D. Edwards, and M. Ramia. “Exploring Serum Biomarkers for Mild Traumatic Brain Injury.” In Brain Neurotrauma: Molecular, Neuropsychological, and Rehabilitation Aspects, edited by F. H. Kobeissy, Frontiers in Neuroengineering. Boca Raton (FL), 2015.
Petzold, A., A. J. Green, G. Keir, S. Fairley, N. Kitchen, M. Smith, and E. J. Thompson. “Role of Serum S100b as an Early Predictor of High Intracranial Pressure and Mortality in Brain Injury: A Pilot Study.” Crit Care Med 30, no. 12 (December 2002): 2705-10. dx.doi.org/10.1097/00003246-200212000-00015.
Qiu, W., C. Guo, H. Shen, K. Chen, L. Wen, H. Huang, M. Ding, L. Sun, Q. Jiang, and W. Wang. “Effects of Unilateral Decompressive Craniectomy on Patients with Unilateral Acute Post-Traumatic Brain Swelling after Severe Traumatic Brain Injury.” Crit Care 13, no. 6 (2009): R185. dx.doi.org/10.1186/cc8178.
Raso Vasquez, A. O., M. D. Kertai, and M. L. Fontes. “Postoperative Thrombocytopenia: Why You Should Consider Antiplatelet Therapy?” Curr Opin Anaesthesiol 31, no. 1 (February 2018): 61-66. dx.doi.org/10.1097/ACO.0000000000000551.
Rothermundt, M., M. Peters, J. H. Prehn, and V. Arolt. “S100b in Brain Damage and Neurodegeneration.” Microsc Res Tech 60, no. 6 (Apr. 15 2003): 614-32. dx.doi.org/10.1002/jemt. 10303.
Rutigliano, D., M. R. Egnor, C. J. Priebe, J. E. McCormack, N. Strong, R. J. Scriven, and T. K. Lee. “Decompressive Craniectomy in Pediatric Patients with Traumatic Brain Injury with Intractable Elevated Intracranial Pressure.” J Pediatr Surg 41, no. 1 (January 2006): 83-7; discussion 83-7. dx.doi.org/10.1016/j.jpedsurg.2005.10.010.
Shim, H. K., S. H. Yu, B. C. Kim, J. H. Lee, and H. J. Choi. “Relationship between Clinical Outcomes and Superior Sagittal Sinus to Bone Flap Distance During Unilateral Decompressive Craniectomy in Patients with Traumatic Brain Injury: Experience at a Single Trauma Center.” Korean J Neurotrauma 14, no. 2 (October 2018): 99-104. dx.doi.org/10.13004/kjnt.2018.14.2.99.
Simon, D. W., M. J. McGeachy, H. Bayir, R. S. Clark, D. J. Loane, and P. M. Kochanek. “The Far-Reaching Scope of Neuroinflammation after Traumatic Brain Injury.” Nat Rev Neurol 13, no. 3 (March 2017): 171-91. dx.doi.org/10.1038/nrneurol.2017.13.
Steiner, J., H. G. Bernstein, H. Bielau, A. Berndt, R. Brisch, C. Mawrin, G. Keilhoff, and B. Bogerts. “Evidence for a Wide Extra-Astrocytic Distribution of S100b in Human Brain.” BMC Neurosci 8 (Jan. 2 2007): 2. dx.doi.org/10.1186/1471-2202-8-2.
Streitburger, D. P., K. Arelin, J. Kratzsch, J. Thiery, J. Steiner, A. Villringer, K. Mueller, and M. L. Schroeter. “Validating Serum S100b and Neuron-Specific Enolase as Biomarkers for the Human Brain—a Combined Serum, Gene Expression and Mri Study.” PLOS One 7, no. 8 (2012): e43284. dx.doi.org/10.1371/journal.pone.0043284.
Su, T. M., C. M. Lan, T. H. Lee, S. W. Hsu, N. W. Tsai, and C. H. Lu. “Risk Factors for the Development of Posttraumatic Hydrocephalus after Unilateral Decompressive Craniectomy in Patients with Traumatic Brain Injury.” J Clin Neurosci 63 (May 2019): 62-67. dx.doi.org/10.1016/j.jocn.2019.02.006.
Tapper, J., M. B. Skrifvars, R. Kivisaari, J. Siironen, and R. Raj. “Primary Decompressive Craniectomy Is Associated with Worse Neurological Outcome in Patients with Traumatic Brain Injury Requiring Acute Surgery.” Surg Neurol Int 8 (2017): 141. dx.doi.org/10.4103/sni.sni 453 16.
Timofeev, I., M. Czosnyka, K. L. Carpenter, J. Nortje, P. J. Kirkpatrick, P. G. Al-Rawi, D. K. Menon, J. D. Pickard, A. K. Gupta, and P. J. Hutchinson. “Interaction between Brain Chemistry and Physiology after Traumatic Brain Injury: Impact of Autoregulation and Microdialysis Catheter Location.” J Neurotrauma 28, no. 6 (June 2011): 849-60. dx.doi.org/10.1089/neu.2010.1656.
Timofeev, I., C. Dahyot-Fizelier, N. Keong, J. Nortje, P. G. Al-Rawi, M. Czosnyka, D. K. Menon, P. J. Kirkpatrick, A. K. Gupta, and P. J. Hutchinson. “Ventriculostomy for Control of Raised Icp in Acute Traumatic Brain Injury.” Acta Neurochir Suppl 102 (2008): 99-104. ncbi.nlm.nih.gov/pubmed/19388297.
Timofeev, I., J. Nortje, P. G. Al-Rawi, P. J. Hutchinson, and A. K. Gupta. “Extracellular Brain Ph with or without Hypoxia Is a Marker of Profound Metabolic Derangement and Increased Mortality after Traumatic Brain Injury.” J Cereb Blood Flow Metab 33, no. 3 (March 2013): 422-7. dx.doi.org/10.1038/jcbfm.2012.186.
Wang, J. C., Q. H. Wang, and J. X. Tian. “Therapeutic Effect of Integrated Traditional Chinese and Western 601 Medicine on Patients with Mental Disorders Caused by Traumatic Brain Injury.” Chin. J. Pract. Nerv. Dis. 18, no. 8 (2015): 50-52.
Xing, Z., Z. Xia, W. Peng, J. Li, C. Zhang, C. Fu, T. Tang, J. Luo, Y. Zou, R. Fan, W. Liu, X. Xiong, W. Huang, C. Sheng, P. Gan, and Y. Wang. “Xuefu Zhuyu Decoction, a Traditional Chinese Medicine, Provides Neuroprotection in a Rat Model of Traumatic Brain Injury Via an Anti-Inflammatory Pathway.” Sci Rep 6 (Jan. 28 2016): 20040. dx.doi.org/10.1038/srep20040.
Yamada, H., Y. Yamada, T. Adachi, A. Fukatsu, M. Sakuma, A. Futenma, and S. Kakumu. “Protective Role of Extracellular Superoxide Dismutase in Hemodialysis Patients.” Nephron 84, no. 3 (March 2000): 218-23. dx.doi.org/10.1159/000045580.
Yunoki, M., M. Kawauchi, N. Ukita, Y. Noguchi, S. Nishio, Y. Ono, S. Asari, T. Ohmoto, M. Asanuma, and N. Ogawa. “Effects of Lecithinized Superoxide Dismutase on Traumatic Brain Injury in Rats.” J Neurotrauma 14, no. 10 (October 1997): 739-46. dx.doi.org/10.1089/neu. 1997.14.739.
Zaghloul, N., H. Patel, C. Codipilly, P. Marambaud, S. Dewey, S. Frattini, P. T. Huerta, M. Nasim, E. J. Miller, and M. Ahmed. “Overexpression of Extracellular Superoxide Dismutase Protects against Brain Injury Induced by Chronic Hypoxia.” PLOS One 9, no. 9 (2014): e108168. dx.doi.org/10.1371/journal.pone.0108168.
Zhang, X., Y. Chen, L. W. Jenkins, P. M. Kochanek, and R. S. Clark. “Bench-to-Bedside Review: Apoptosis/Programmed Cell Death Triggered by Traumatic Brain Injury.” Crit Care 9, no. 1 (February 2005): 66-75. dx.doi.org/10.1186/cc2950.
Zhou, M., R. Dominguez, and M. Baudry. “Superoxide Dismutase/Catalase Mimetics but Not Map Kinase Inhibitors Are Neuroprotective against Oxygen/Glucose Deprivation-Induced Neuronal Death in Hippocampus.” J Neurochem 103, no. 6 (December 2007): 2212-23. dx.doi.org/10.1111/j.1471-4159.2007.04906.x.

TABLE 1
Clinical characteristics, GCS score, causes and subtypes of TBI of the participants at admission

| Characteristics/No. of cases | OT (n = 20) | XNJ (n = 20) | l-YB (n = 20) | h-YB (n = 20) | X² | P |
|---|---|---|---|---|---|---|
| Male:Female | 12:8 | 11:9 | 13:7 | 12:8 | 0.417 | 0.937 ^{a} |
| Age, y, mean(min-max) | 41.7 ± 10.54 | 43.1 ± 10.66 | 42.3 ± 14.05 | 42.3 ± 9.65 | 0.461 | 0.927 ^{b} |
| Degree of injury | | | | | | |
| Severe:Moderate | 10:10 | 8:12 | 8:12 | 12:8 | 2.020 | 0.568 ^{a} |
| Admission time (hours from injury) | 4.7(1.5-8) | 5.3(2.5-12) | 4.8(2-7.5) | 5.1(2-8) | 0.59 | 0.899 ^{b} |
| mean(min-max) | | | | | | |
| Causes of TBI (N/%) | | | | | 3.628 | 0.989 ^{a} |
| Traffic accident | 9(45) | 7(35) | 7(35) | 8(40) | | |
| Falling | 4(20) | 5(25) | 4(20) | 3(15) | | |
| Blow | 5(25) | 3(15) | 4(20) | 4(20) | | |
| tumble | 2(10) | 3(15) | 3(15) | 3(15) | | |
| crush | 0(0) | 2(10) | 2(10) | 2(10) | | |
| Damage type (N/%) | | | | | 2 296 | 0.999^{a} |
| Epidural hematoma | 2(10) | 3(15) | 2(10) | 3(15) | | |
| Subdural hematoma | 5(25) | 6(30) | 6(30) | 4(20) | | |
| Brain contusion | 10(50) | 9(45) | 9(50) | 10(50) | | |
| Intracerebral hematoma | 2(10) | 1(5) | 1(5) | 1(5) | | |
| Multiple hematoma | 1(5) | 1(5) | 2(10) | 2(10) | | |
| Operation method (N/%) | | | | | | |
| Decompressive craniectomy | 12(60) | 8(40) | 9(45) | 11(55) | 2.000 | 0.572 ^{a} |
| Intracranial hematoma evacuation | 10(50) | 11(55) | 11(55) | 10(50) | 0.201 | 0.978 ^{a} |
| Inactivated brain tissue resection | 10(50) | 9(45) | 9(45) | 10(50) | 0.201 | 0.978 ^{a} |

^{a} Chi-square Test;
^{b} K Independent Samples test.
OT, Orthodox therapy group;
XNJ, OT + Xingnaojing group;
l-YB, OT + low-dose Yunnan Baiyao group;
h-YB, OT + high dose Yunnan Baiyao group

TABLE 2
Repeated measure analysis of Changes in GCS, S100B,
SOD, Glasgow Outcome Scale and Karnofsky Performance
Scale after emergency craniotomy in patients with
moderate-to severe TBI show significant differences
in therapeutic effects between OT, XNJ, I-YB and h-YB
and time * treatment interaction of, therapies

| | | | Time * |
|---|---|---|---|
| No. of | Treatment | Time | Treatment |
| cases | F, P | F, P | F, P |
| GCS (N, 80) | 1.57, | 250.8, | 6.16, |
| | 0.20 | 0.00** | 0.00** |
| S100B (N, 80) | 40.4, | 1482.8, | 27.3, |
| | 0.000** | 0.00** | 0.00** |
| SOD (N, 80) | 19.1, | 456.1, | 22.8, |
| | 0.000** | 0.00** | 0.00** |
| GOS (N,80) | 4.60, | 138.6, | 0.839, |
| | 0.005** | 0.00** | 0.477 |
| KPS (N, 80) | 7.04, | 378.0, | 0.489, |
| | 0.000** | 0.00** | 0.691 |

GCS, Glasgow Coma Scale
GOS, Glasgow Outcome Scale
KPS, Karnofsky Performance Scale
S100B, serum S100B level
SOD, serum superoxide dismutase activity
**P < 0.01, vs. different therapies, or vs. different observation time

TABLE 3
Changes in GCS, S100B, SOD, GOS and KPS in patients with moderate-to-severe TBI and craniotomy after orthodox therapy,
Xingnaojing, low and high doses of Yunnan Baiyao adjunct therapies (means ± SD)

| | | OT (n = 20) | | OT + XNJ (n = 20) | | OT + l-YB (n = 20) | | OT + h-YB (n = 20) | |
|---|---|---|---|---|---|---|---|---|---|
| | | | P ^{(a)} | | P ^{(a)}, P ^{(b)} | | P ^{(a)}, P ^{(b)} | | P ^{(a)}, P ^{(b)} |
| | Days | | | | | | | | |
| GCS Score | Admission | 8.10 ± 2.38 | / | 8.60 ± 2.50 | /, 0.52 | 8.50 ± 2 48 | /, 0.61 | 7.30 ± 2.43 | /, 0.31 |
| | 1 | 8.20 ± 2.29 | 0.063 | 8.80 ± 2.33 | 0.059, 0.40 | 8.70 ± 2.16 | 0.061, 0.48 | 7.35 ± 2.16 | 0.057, 0.23 |
| | 3 | 8.30 ± 1.95 | 0.056 | 9.90 ± 2.00 | 0.018, 0.01 | 9.90 ± 1.97 | 0.016, 0.01 | 9.80 ± 1.77 | 0.022, 0.02 |
| | 5 | 9.35 ± 2.06 | 0.029 | 10.90 ± 1.58 | <0.001, 0.01 | 10.65 ± 1.69 | <0.001, 0.02 | 10.95 ± 1.43 | <0.001, <0.001 |
| | 7 | 10.10 ± 1.80 | 0.021 | 11.85 ± 1.93 | <0.001, | 11.55 ± 1.70 | <0.001, 0.01 | 11.30 ± 1.53 | <0.001, 0.03 |
| | | | | | <0.001 | | | | |
| S100B | Admission | 2.08 ± 0.14 | / | 1.99 ± 0.18 | 0.07 | 2.00 ± 0.15 | 0.11 | 2.00 ± 0.16 | 0.09 |
| protein | 1 | 2.74 ± 0.15 | | 2.67 ± 0.13 | 0.13 | 2.81 ± 0.14 | 0.11 | 2.73 ± 0.14 | 0.89 |
| (μg/l) | 3 | 3.38 ± 0.23 | | 2.99 ± 0.34 | <0.001 | 3.20 ± 0.32 | 0.05 | 3.12 ± 0.30 | 0.01 |
| | 5 | 2.84 ± 0.20 | | 1.83 ± 0.23 | <0.001 | 1.93 ± 0.29 | <0.001 | 1.94 ± 0.18 | <0.001 |
| | 7 | 2.48 ± 0.23 | | 1.57 ± 0.29 | <0.001 | 1.59 ± 0.23 | <0.001 | 1.51 ± 0.25 | <0.001 |
| Serum SOD | Admission | 114.80 ± 8.93 | / | 112.91 ± 8.61 | 0.47 | 115.44 ± 7.23 | 0.81 | 115.76 ± 7.77 | 0.71 |
| activity | 1 | 105.10 ± 7.95 | | 105.65 ± 9.03 | 0.83 | 101.06 ± 6.15 | 0.12 | 103.05 ± 8.67 | 0.42 |
| (U/ml) | 3 | 74.75 ± 7.51 | | 95.26 ± 6.00 | <0.001 | 91.79 ± 6.64 | <0.001 | 94.41 ± 6.07 | <0.001 |
| | 5 | 80.97 ± 5.85 | | 104.60 ± 7.89 | <0.001 | 101.64 ± 7.92 | <0.001 | 101.74 ± 5.82 | <0.001 |
| | 7 | 83.47 ± 7.71 | | 112.59 ± 9.83 | <0.001 | 107.24 ± 9.23 | <0.001 | 110.29 ± 6.05 | <0.001 |
| | Months | | | | | | | | |
| GOS Score | 1 | 2.90 ± 1.07 | / | 3.80 ± 0.95 | /, 0.00 | 3.70 ± 0.57 | /, 0.01 | 3.70 ± 0.87 | /, 0.01 |
| KPS Score | 3 | 3.65 ± 0.99 | / | 4.35 ± 0.81 | /, 0.01 | 4.45 ± 0.61 | /, <0.001 | 4.30 ± 0.80 | /, 0.01 |
| | 1 | 39.00 ± 12.25 | / | 62.50 ± 20.49 | /, <0.001 | 59.50 ± 10.99 | /, <0.001 | 58.50 ± 17.25 | /, <0.001 |
| | 3 | 57.00 ± 20.80 | / | 78.00 ± 18.53 | /, <0.001 | 77.50 ± 15.17 | /, <0.001 | 76.50 ± 17.55 | /, <0.001 |
| Change in | | | | | | | | | |
| Score Score | | | | | | | | | |
| | Days | | | | | | | | |
| GCS score | 1 | 0.10 ± 0.85 | 0.063 | 0.20 ± 0.89 | 0.059, 0.72 | 0.20 ± 0.89 | 0.061, 0.72 | 0.05 ± 0.89 | 0.057, 0.86 |
| | 3 | 0.20 ± 1.36 | 0.056 | 1.30 ± 0.73 | 0.018, <0.001 | 1.40 ± 0.82 | 0.016, <0.001 | 2.50 ± 1.32 | 0.022, <0.001 |
| | 5 | 1.25 ± 0.79 | 0.029 | 2.30 ± 1.22 | <0.001, 0.003 | 2.15 ± 0.93 | <0.001, 0.01 | 3.65 ± 1.35 | <0.001, <0.001 |
| | 7 | 2.00 ± 0.86 | 0.021 | 3.25 ± 1.02 | <0.001, | 3.05 ± 1.00 | <0.001, 0.002 | 4.00 ± 1.17 | <0.001, <0.001 |
| | | | | | <0.001 | | | | |
| Change in | 1 | 0.65 ± 0.17 | | 0.68 ± 0.19 | 0.57 | 0.80 ± 0.10 | 0.002 | 0.73 ± 0.09 | 0.09 |
| S100B | 3 | 1.30 ± 0.18 | | 0.99 ± 0.29 | <0.001 | 1.19 ± 0.26 | 0.17 | 1.12 ± 0.21 | 0.02 |
| (μg/l) | 5 | 0.75 ± 0.19 | | −0.16 ± 0.25 | <0.001 | −0.07 ± 0.24 | <0.001 | −0.06 ± 0.16 | <0.001 |
| | 7 | 0.40 ± 0.23 | | −0.42 ± 0.33 | <0.001 | −0.42 ± 0.10 | <0.001 | −0.49 ± 0.13 | <0.001 |
| Change in | 1 | −9.70 ± 9.24 | | −7.26 ± 5.54 | 0.17 | −14.38 ± 1.38 | 0.009 | −12.71 ± | 0.09 |
| SOD (U/ml) | | | | | | | | 12.63 | |
| | 3 | −40.05 ± 9.22 | | −17.65 ± 4.72 | <0.001 | −23.65 ± 3.97 | <0.001 | −0.49 ± 2.56 | <0.001 |
| | 5 | −33.83 ± | | −8.31 ± 5.62 | <0.001 | −13.80 ± 4.09 | <0.001 | −12.71 ± 2.07 | <0.001 |
| | | 11.04 | | | | | | | |
| | 7 | −31.33 ± | | −0.33 ± 5.42 | <0.001 | −8.20 ± 3.56 | <0.001 | −5.47 ± 2.07 | <0.001 |
| | | 11.44 | | | | | | | |
| | Months | | | | | | | | |
| Change in | 3 | 0.75 ± 0.44 | / | 0.55 ± 0.61 | /, 0.21 | 0.75 ± 0.44 | /, 1.00 | 0.60 ± 0.50 | /, 0.35 |
| GOS | | | | | | | | | |
| Change in | 3 | 18.00 ± 7.68 | / | 15.50 ± 8.26 | /, 0.33 | 18.00 ± 8.34 | /, 1.00 | 18.00 ± 7.68 | /, 1.00 |
| KPS | | | | | | | | | |
| | Days | | | | | | | | |
| (%) Change | 1 | 1.98 ± 10.72 | 0.063 | 4.02 ± 12.20 | 0.059, 0.60 | 4.48 ± 11.93 | 0.061, 0.52 | 2.58 ± 13.73 | 0.057, 0.88 |
| in GCS | 3 | 5.29 ± 16.70 | 0.056 | 18.76 ± 15.55 | 0.018, 0.06 | 21.09 ± 20.46 | 0.016, 0.03 | 42.67 ± 32.53 | 0.022, <0 001 |
| | 5 | 18.31 ± 13.55 | 0.029 | 33.58 ± 27.70 | <0.001, 0.09 | 31.62 ± 25.15 | <0.001, 0.14 | 61.37 ± 39.55 | <0.001, <0.001 |
| | 7 | 29.47 ± 20.63 | 0.021 | 44.36 ± 28.26 | <0.001, 0.12 | 43.52 ± 30.33 | <0.001, 0.14 | 65.83 ± 38.00 | <0.001, <0.001 |
| (%) Change | 1 | 31.68 ± 9.41 | | 34.94 ± 12.50 | 0.26 | 40.43 ± 6.75 | 0.003 | 36.94 ± 6.58 | 0.07 |
| in S100B | 3 | 62.49 ± 9.51 | | 50.56 ± 15.97 | 0.004 | 59.71 ± 13.63 | 0.49 | 56.19 ± 9.98 | 0.12 |
| | 5 | 36.49 ± 9.82 | | −7.70 ± 13.02 | <0.001 | −3.73 ± 12.23 | <0.001 | −2.92 ± 7.97 | <0.001 |
| | 7 | 19.44 ± 11.29 | | −20.51 ± 16.17 | <0.001 | −21.12 ± 6.33 | <0.001 | −24.71 ± 8.24 | <0.001 |
| (%) Change | 1 | −9.56 ± 8.93 | | −7.08 ± 5.49 | 0.15 | −14.22 ± 0.92 | 0.008 | −12.50 ± 2.58 | 0.09 |
| in SOD | 3 | −38.30 ± 8.76 | | −16.73 ± 4.32 | <0.001 | −23.40 ± 3.71 | <0.001 | −20.71 ± 1.60 | <0.001 |
| | 5 | −32.22 ± | | −7.99 ± 5.45 | <0.001 | −13.69 ± 4.10 | <0.001 | −13.55 ± 1.08 | <0.001 |
| | | 10.00 | | | | | | | |
| | 7 | −29.81 ± | | −0.52 ± 5.15 | <0.001 | −8.22 ± 3.75 | <0.001 | −5.20 ± 1.61 | <0.001 |
| | | 10.66 | | | | | | | |
| | Months | | | | | | | | |
| (%) Change | 3 | 30.00 ± 19.76 | / | 17.50 ± 20.21 | /, 0.03 | 21.25 ± 13.10 | /, 0.12 | 18.33 ± 16.36 | /, 0.04 |
| in GOS | | | | | | | | | |
| (%) Change | 3 | 62.90 ± 43.30 | / | 29.93 ± 21.01 | /, <0.001 | 30.58 ± 14.56 | /, <0.001 | 34.65 ± 18.76 | /, 0.001 |
| in KPS | | | | | | | | | |

P ^{(a)}, P values, compared with the value at admission
P ^{(b)} P values, compared with the value of OT

TABLE 4
Correlations between the acute and long-term variables of TBI

| | | GCSa | GCS1 | GCS3 | GCS5 | GCS7 | GOS1 | GOS3 |
|---|---|---|---|---|---|---|---|---|
| GCSa | Pearson | 1 | .936** | .835** | .835** | .875** | .689** | .685** |
| | Correlation | | | | | | | |
| | Sig. (2- | 0.000 | 0.000 | 0.000 | 0.000 | 0.000 | 0.000 | 0.000 |
| | tailed) | | | | | | | |
| | N | 80 | 80 | 80 | 80 | 80 | 80 | 80 |
| GCS1 | Pearson | .936** | 1 | .847** | .808** | .860** | .600** | .619** |
| | Correlation | | | | | | | |
| | Sig. (2- | 0.000 | | 0.000 | 0.000 | 0.000 | 0.000 | 0.000 |
| | tailed) | | | | | | | |
| | N | 80 | 80 | 80 | 80 | 80 | 80 | 80 |
| GCS3 | Pearson | .835** | .847** | 1 | .935** | .925** | .711** | .675** |
| | Correlation | | | | | | | |
| | Sig. (2- | 0.000 | 0.000 | | 0.000 | 0.000 | 0.000 | 0.000 |
| | tailed) | | | | | | | |
| | N | 80 | 80 | 80 | 80 | 80 | 80 | 80 |
| GCS5 | Pearson | .835** | .808** | .935** | 1 | .932** | .768** | .714** |
| | Correlation | | | | | | | |
| | Sig. (2- | 0.000 | 0.000 | 0.000 | | 0.000 | 0.000 | 0.000 |
| | tailed) | | | | | | | |
| | N | 80 | 80 | 80 | 80 | 80 | 80 | 80 |
| GCS7 | Pearson | .875** | .860** | .925** | .932** | 1 | .788** | .738** |
| | Correlation | | | | | | | |
| | Sig. (2- | 0.000 | 0.000 | 0.000 | 0.000 | | 0.000 | 0.000 |
| | tailed) | | | | | | | |
| | N | 80 | 80 | 80 | 80 | 80 | 80 | 80 |
| GOS1 | Pearson | .689** | .600** | .711** | .768** | .788** | 1 | .848** |
| | Correlation | | | | | | | |
| | Sig. (2- | 0.000 | 0.000 | 0.000 | 0.000 | 0.000 | | 0.000 |
| | tailed) | | | | | | | |
| | N | 80 | 80 | 80 | 80 | 80 | 80 | 80 |
| GOS3 | Pearson | .685** | .619** | .675** | .714** | .738** | .848** | 1 |
| | Correlation | | | | | | | |
| | Sig. (2- | 0.000 | 0.000 | 0.000 | 0.000 | 0.000 | 0.000 | |
| | tailed) | | | | | | | |
| | N | 80 | 80 | 80 | 80 | 80 | 80 | 80 |
| KPS1 | Pearson | .688** | .605** | .720** | .764** | .799** | .968** | .878** |
| | Correlation | | | | | | | |
| | Sig. (2- | 0.000 | 0.000 | 0.000 | 0.000 | 0.000 | 0.000 | 0.000 |
| | tailed) | | | | | | | |
| | N | 80 | 80 | 80 | 80 | 80 | 80 | 80 |
| KPS3 | Pearson | .694** | .614** | .713** | .758** | .781** | .877** | .962** |
| | Correlation | | | | | | | |
| | Sig. (2- | 0.000 | 0.000 | 0.000 | 0.000 | 0.000 | 0.000 | 0.000 |
| | tailed) | | | | | | | |
| | N | 80 | 80 | 80 | 80 | 80 | 80 | 80 |
| S100a | Pearson | −.354** | −.281* | −.398** | −.433** | −.434** | −.438** | −.412** |
| | Correlation | | | | | | | |
| | Sig. (2- | 0.001 | 0.012 | 0.000 | 0.000 | 0.000 | 0.000 | 0.000 |
| | tailed) | | | | | | | |
| | N | 80 | 80 | 80 | 80 | 80 | 80 | 80 |
| S1001 | Pearson | −.399** | −.316** | −.382** | −.401** | −.431** | −.396** | −.337** |
| | Correlation | | | | | | | |
| | Sig. (2- | 0.000 | 0.004 | 0.000 | 0.000 | 0.000 | 0.000 | 0.002 |
| | tailed) | | | | | | | |
| | N | 80 | 80 | 80 | 80 | 80 | 80 | 80 |
| S1003 | Pearson | −.324** | −.289** | −.353** | −.380** | −.376** | −.414** | −.387** |
| | Correlation | | | | | | | |
| | Sig. (2- | 0.003 | 0.009 | 0.001 | 0.001 | 0.001 | 0.000 | 0.000 |
| | tailed) | | | | | | | |
| | N | 80 | 80 | 80 | 80 | 80 | 80 | 80 |
| S1005 | Pearson | −0.146 | −0.125 | −.423** | −.419** | −.427** | −.456** | −.483** |
| | Correlation | | | | | | | |
| | Sig. (2- | 0.197 | 0.269 | 0.000 | 0.000 | 0.000 | 0.000 | 0.000 |
| | tailed) | | | | | | | |
| | N | 80 | 80 | 80 | 80 | 80 | 80 | 80 |
| S1007 | Pearson | −.333** | −.283* | −.567** | −.627** | −.630** | −.606** | −.535** |
| | Correlation | | | | | | | |
| | Sig. (2- | 0.003 | 0.011 | 0.000 | 0.000 | 0.000 | 0.000 | 0.000 |
| | tailed) | | | | | | | |
| | N | 80 | 80 | 80 | 80 | 80 | 80 | 80 |
| SODa | Pearson | .329** | .381** | .390** | .361** | .346** | .250* | 327** |
| | Correlation | | | | | | | |
| | Sig. (2- | 0.003 | 0.000 | 0.000 | 0.001 | 0.002 | 0.025 | 0.003 |
| | tailed) | | | | | | | |
| | N | 80 | 80 | 80 | 80 | 80 | 80 | 80 |
| SOD1 | Pearson | .335** | .347** | .324** | .28** | .313** | .270* | .249* |
| | Correlation | | | | | | | |
| | Sig. (2- | 0.002 | 0.002 | 0.003 | 0.010 | 0.005 | 0.016 | 0.026 |
| | tailed) | | | | | | | |
| | N | 80 | 80 | 80 | 80 | 80 | 80 | 80 |
| SOD3 | Pearson | 0.213 | .244* | .495** | .523** | .507** | .492** | .485** |
| | Correlation | | | | | | | |
| | Sig. (2- | 0.058 | 0.029 | 0.000 | 0.000 | 0.000 | 0.000 | 0.000 |
| | tailed) | | | | | | | |
| | N | 80 | 80 | 80 | 80 | 80 | 80 | 80 |
| SOD5 | Pearson | 0.189 | 0.192 | .436** | .455** | .478** | .477** | .418** |
| | Correlation | | | | | | | |
| | Sig. (2- | 0.093 | 0.087 | 0.000 | 0.000 | 0.000 | 0.000 | 0.000 |
| | tailed) | | | | | | | |
| | N | 80 | 80 | 80 | 80 | 80 | 80 | 80 |
| SOD7 | Pearson | .260* | .260* | .535** | .551** | .553** | .540** | .467** |
| | Correlation | | | | | | | |
| | Sig. (2- | 0.020 | 0.020 | 0.000 | 0.000 | 0.000 | 0.000 | 0.000 |
| | tailed) | | | | | | | |
| | N | 80 | 80 | 80 | 80 | 80 | 80 | 80 |
| | | KPS1 | KPS3 | S100a | S1001 | S1003 | S1005 | S1007 |
| GCSa | Pearson | .688** | .694** | −.354** | −.399** | .324** | −0.146 | −.333** |
| | Correlation | | | | | | | |
| | Sig. (2- | 0.000 | 0.000 | 0.001 | 0.000 | 0.003 | 0.197 | 0.003 |
| | tailed) | | | | | | | |
| | N | 80 | 80 | 80 | 80 | 80 | 80 | 80 |
| GCS1 | Pearson | .605** | .614** | −.281* | −.316** | −.289** | −0.125 | −.283* |
| | Correlation | | | | | | | |
| | Sig. (2- | 0.000 | 0.000 | 0.012 | 0.004 | 0.009 | 0.269 | 0.011 |
| | tailed) | | | | | | | |
| | N | 80 | 80 | 80 | 80 | 80 | 80 | 80 |
| GCS3 | Pearson | .720** | .713** | −.398** | −.382** | −.353** | −.423** | −.567** |
| | Correlation | | | | | | | |
| | Sig. (2- | 0.000 | 0.000 | 0.000 | 0.000 | 0.001 | 0.000 | 0.000 |
| | tailed) | | | | | | | |
| | N | 80 | 80 | 80 | 80 | 80 | 80 | 80 |
| GCS5 | Pearson | .764** | .758** | −.433** | −.401** | −.380** | −.419** | −.627** |
| | Correlation | | | | | | | |
| | Sig. (2- | 0.000 | 0.000 | 0.000 | 0.000 | 0.001 | 0.000 | 0.000 |
| | tailed) | | | | | | | |
| | N | 80 | 80 | 80 | 80 | 80 | 80 | 80 |
| GCS7 | Pearson | .799** | .781** | −434** | −.431** | −.376** | −.427** | −.630** |
| | Correlation | | | | | | | |
| | Sig. (2- | 0.000 | 0.000 | 0.000 | 0.000 | 0.001 | 0.000 | 0.000 |
| | tailed) | | | | | | | |
| | N | 80 | 80 | 80 | 80 | 80 | 80 | 80 |
| GOS1 | Pearson | .968** | .877** | −.438** | −.396** | −.414** | −.456** | −.606** |
| | Correlation | | | | | | | |
| | Sig. (2- | 0.000 | 0.000 | 0.000 | 0.000 | 0.000 | 0.000 | 0.000 |
| | tailed) | | | | | | | |
| | N | 80 | 80 | 80 | 80 | 80 | 80 | 80 |
| GOS3 | Pearson | .878** | .962** | −.412** | −.337** | −.387** | −.483** | −.535** |
| | Correlation | | | | | | | |
| | Sig. (2- | 0.000 | 0.000 | 0.000 | 0.002 | 0.000 | 0.000 | 0.000 |
| | tailed) | | | | | | | |
| | N | 80 | 80 | 80 | 80 | 80 | 80 | 80 |
| KPS1 | Pearson | 1 | .921** | −.487** | −.402** | −.446** | −.544** | −.655** |
| | Correlation | | | | | | | |
| | Sig. (2- | | 0.000 | 0.000 | 0.000 | 0.000 | 0.000 | 0.000 |
| | tailed) | | | | | | | |
| | N | 80 | 80 | 80 | 80 | 80 | 80 | 80 |
| KPS3 | Pearson | .921** | 1 | −.469** | −.355** | −.429** | −.527** | −.616** |
| | Correlation | | | | | | | |
| | Sig. (2- | 0.000 | | 0.000 | 0.001 | 0.000 | 0.000 | 0.000 |
| | tailed) | | | | | | | |
| | N | 80 | 80 | 80 | 80 | 80 | 80 | 80 |
| S100a | Pearson | −.487** | −.469** | 1 | .502** | .638** | .444** | .493** |
| | Correlation | | | | | | | |
| | Sig. (2- | 0.000 | 0.000 | | 0.000 | 0.000 | 0.000 | 0.000 |
| | tailed) | | | | | | | |
| | N | 80 | 80 | 80 | 80 | 80 | 80 | 80 |
| S1001 | Pearson | −.402** | −.355** | .502** | 1 | .405** | 0.200 | .314** |
| | Correlation | | | | | | | |
| | Sig. (2- | 0.000 | 0.001 | 0.000 | | 0.000 | 0.075 | 0.005 |
| | tailed) | | | | | | | |
| | N | 80 | 80 | 80 | 80 | 80 | 80 | 80 |
| S1003 | Pearson | −.446** | −.429** | .638** | .405** | 1 | .517** | .526** |
| | Correlation | | | | | | | |
| | Sig. (2- | 0.000 | 0.000 | 0.000 | 0.000 | | 0.000 | 0.000 |
| | tailed) | | | | | | | |
| | N | 80 | 80 | 80 | 80 | 80 | 80 | 80 |
| S1005 | Pearson | −.544** | −.527** | .444** | 0.200 | .517** | 1 | .810** |
| | Correlation | | | | | | | |
| | Sig. (2- | 0.000 | 0.000 | 0.000 | 0.075 | 0.000 | | 0.000 |
| | tailed) | | | | | | | |
| | N | 80 | 80 | 80 | 80 | 80 | 80 | 80 |
| S1007 | Pearson | −655** | −.616** | .493** | .314** | .526** | .810** | 1 |
| | Correlation | | | | | | | |
| | Sig. (2- | 0.000 | 0.000 | 0.000 | 0.005 | 0.000 | 0.000 | |
| | tailed) | | | | | | | |
| | N | 80 | 80 | 80 | 80 | 80 | 80 | 80 |
| SODa | Pearson | .230* | .336** | −0.027 | 0.039 | −0.037 | 0.015 | −0.057 |
| | Correlation | | | | | | | |
| | Sig. (2- | 0.040 | 0.002 | 0.814 | 0.728 | 0.747 | 0.894 | 0.618 |
| | tailed) | | | | | | | |
| | N | 80 | 80 | 80 | 80 | 80 | 80 | 80 |
| SOD1 | Pearson | .249* | .296** | 0.075 | −0.087 | 0.026 | 0.102 | 0.030 |
| | Correlation | | | | | | | |
| | Sig. (2- | 0.026 | 0.008 | 0.510 | 0.442 | 0.818 | 0.367 | 0.790 |
| | tailed) | | | | | | | |
| | N | 80 | 80 | 80 | 80 | 80 | 80 | 80 |
| SOD3 | Pearson | .532** | .561** | −.244* | −0.091 | −.396** | −.691** | −.718** |
| | Correlation | | | | | | | |
| | Sig. (2- | 0.000 | 0.000 | 0.029 | 0.423 | 0.000 | 0.000 | 0.000 |
| | tailed) | | | | | | | |
| | N | 80 | 80 | 80 | 80 | 80 | 80 | 80 |
| SOD5 | Pearson | .527** | .519** | −0.158 | −0.075 | −.304** | −.669** | −.709** |
| | Correlation | | | | | | | |
| | Sig. (2- | 0.000 | 0.000 | 0.162 | 0.510 | 0.006 | 0.000 | 0.000 |
| | tailed) | | | | | | | |
| | N | 80 | 80 | 80 | 80 | 80 | 80 | 80 |
| SOD7 | Pearson | .581** | .569** | −.224* | −0.154 | −.363** | −.695** | −.750** |
| | Correlation | | | | | | | |
| | Sig. (2- | 0.000 | 0.000 | 0.046 | 0.172 | 0.001 | 0.000 | 0.000 |
| | tailed) | | | | | | | |
| | N | 80 | 80 | 80 | 80 | 80 | 80 | 80 |
| | | | | SODa | SOD1 | SOD3 | SOD5 | SOD7 |
| | | GCSa | Pearson | .329** | .335** | 0.213 | 0.189 | .260* |
| | | | Correlation | | | | | |
| | | | Sig. (2- | 0.003 | 0.002 | 0.058 | 0.093 | 0.020 |
| | | | tailed) | | | | | |
| | | | N | 80 | 80 | 80 | 80 | 80 |
| | | GCS1 | Pearson | .381** | .347** | .244* | 0.192 | .260* |
| | | | Correlation | | | | | |
| | | | Sig. (2- | 0.000 | 0.002 | 0.029 | 0.087 | 0.020 |
| | | | tailed) | | | | | |
| | | | N | 80 | 80 | 80 | 80 | 80 |
| | | GCS3 | Pearson | .390** | .324** | .495** | .436** | .535** |
| | | | Correlation | | | | | |
| | | | Sig. (2- | 0.000 | 0.003 | 0.000 | 0.000 | 0.000 |
| | | | tailed) | | | | | |
| | | | N | 80 | 80 | 80 | 80 | 80 |
| | | GCS5 | Pearson | .361** | .288** | .523** | .455** | .551** |
| | | | Correlation | | | | | |
| | | | Sig. (2- | 0.001 | 0.010 | 0.000 | 0.000 | 0.000 |
| | | | tailed) | | | | | |
| | | | N | 80 | 80 | 80 | 80 | 80 |
| | | GCS7 | Pearson | .346** | .313** | .507** | .478** | .553** |
| | | | Correlation | | | | | |
| | | | Sig. (2- | 0.002 | 0.005 | 0.000 | 0.000 | 0.000 |
| | | | tailed) | | | | | |
| | | | N | 80 | 80 | 80 | 80 | 80 |
| | | GOS1 | Pearson | .250* | .270* | .492** | .477** | .540** |
| | | | Correlation | | | | | |
| | | | Sig. (2- | 0.025 | 0.016 | 0.000 | 0.000 | 0.000 |
| | | | tailed) | | | | | |
| | | | N | 80 | 80 | 80 | 80 | 80 |
| | | GOS3 | Pearson | .327** | .249* | .485** | .418** | .467** |
| | | | Correlation | | | | | |
| | | | Sig. (2- | 0.003 | 0.026 | 0.000 | 0.000 | 0.000 |
| | | | tailed) | | | | | |
| | | | N | 80 | 80 | 80 | 80 | 80 |
| | | KPS1 | Pearson | .230* | .249* | .532** | .527** | .581** |
| | | | Correlation | | | | | |
| | | | Sig. (2- | 0.040 | 0.026 | 0.000 | 0.000 | 0.000 |
| | | | tailed) | | | | | |
| | | | N | 80 | 80 | 80 | 80 | 80 |
| | | KPS3 | Pearson | .336** | .296** | .561** | .519** | .569** |
| | | | Correlation | | | | | |
| | | | Sig. (2- | 0.002 | 0.008 | 0.000 | 0.000 | 0.000 |
| | | | tailed) | | | | | |
| | | | N | 80 | 80 | 80 | 80 | 80 |
| | | S100a | Pearson | −0.027 | 0.075 | −.244* | −0.158 | −.244* |
| | | | Correlation | | | | | |
| | | | Sig. (2- | 0.814 | 0.510 | 0.029 | 0.162 | 0.046 |
| | | | tailed) | | | | | |
| | | | N | 80 | 80 | 80 | 80 | 80 |
| | | S1001 | Pearson | 0.039 | −0.087 | −0.091 | −0.075 | −0.154 |
| | | | Correlation | | | | | |
| | | | Sig. (2- | 0.728 | 0.442 | 0.423 | 0.510 | 0.172 |
| | | | tailed) | | | | | |
| | | | N | 80 | 80 | 80 | 80 | 80 |
| | | S1003 | Pearson | −0.037 | 0.026 | −.396** | −.304** | −.363** |
| | | | Correlation | | | | | |
| | | | Sig. (2- | 0.747 | 0.818 | 0.000 | 0.006 | 0.001 |
| | | | tailed) | | | | | |
| | | | N | 80 | 80 | 80 | 80 | 80 |
| | | S1005 | Pearson | 0.015 | 0.102 | −.691** | −.669** | −.695** |
| | | | Correlation | | | | | |
| | | | Sig. (2- | 0.894 | 0.367 | 0.000 | 0.000 | 0.000 |
| | | | tailed) | | | | | |
| | | | N | 80 | 80 | 80 | 80 | 80 |
| | | S1007 | Pearson | −0.057 | 0.030 | −.718** | −.709** | −.750** |
| | | | Correlation | | | | | |
| | | | Sig. (2- | 0.618 | 0.790 | 0.000 | 0.000 | 0.000 |
| | | | tailed) | | | | | |
| | | | N | 80 | 80 | 80 | 80 | 80 |
| | | SODa | Pearson | 1 | .719** | .421** | .344** | .364** |
| | | | Correlation | | | | | |
| | | | Sig. (2- | | 0.000 | 0.000 | 0.002 | 0.001 |
| | | | tailed) | | | | | |
| | | | N | 80 | 80 | 80 | 80 | 80 |
| | | SOD1 | Pearson | .719** | 1 | .405** | .385** | .365** |
| | | | Correlation | | | | | |
| | | | Sig. (2- | 0.000 | | 0.000 | 0.000 | 0.001 |
| | | | tailed) | | | | | |
| | | | N | 80 | 80 | 80 | 80 | 80 |
| | | SOD3 | Pearson | .421** | .405** | 1 | .904** | .926** |
| | | | Correlation | | | | | |
| | | | Sig. (2- | 0.000 | 0.000 | | 0.000 | 0.000 |
| | | | tailed) | | | | | |
| | | | N | 80 | 80 | 80 | 80 | 80 |
| | | SOD5 | Pearson | .344** | .385** | .904** | 1 | .928** |
| | | | Correlation | | | | | |
| | | | Sig. (2- | 0.002 | 0.000 | 0.000 | | 0.000 |
| | | | tailed) | | | | | |
| | | | N | 80 | 80 | 80 | 80 | 80 |
| | | SOD7 | Pearson | .364** | .365** | .926** | .928** | 1 |
| | | | Correlation | | | | | |
| | | | Sig. (2- | 0.001 | 0.001 | 0.000 | 0.000 | |
| | | | tailed) | | | | | |
| | | | N | 80 | 80 | 80 | 80 | 80 |

1. A method of treating a patient with moderate-to-severee traumatic brain injury (TBI) undergoing emergency craniotomy, the method comprising administering, in addition to orthodox therapy (OT), intravenous Xingnaojing (XNJ) for seven consecutive days in an intensive care setting, wherein acute postoperative neurological recovery is improved as measured by Glasgow Coma Scale (GCS) during postoperative Days 1, 3, 5, and 7, wherein long-term functional outcome is improved as measured by Glasgow Outcome Scale (GOS) and Karnofsky Performance Status (KPS) at 30 and 90 days, and wherein secondary-injury biomarkers are modulated by reducing serum S100B and preserving or restoring serum superoxide dismutase (SOD) activity relative to OT alone.

2. The method of claim 1, wherein the patient meets all of: (a) GCS 3-12 within 12 hours of TBI; (b) computed tomography (CT)-confirmed intracranial lesion; (c) age 15-65 years; and (d) emergency craniotomy within 12 hours of TBI with informed consent.

3. The method of claim 1, wherein the orthodox therapy comprises one or more of dehydration therapy, hemostasis, diuresis, prophylactic antibiotics, hormone therapy, seizure prophylaxis, and supportive care, with daily monitoring of vital signs, consciousness, and pupil reactivity.

4. The method of claim 1, wherein the craniotomy comprises hematoma evacuation, resection of nonviable tissue, bone-flap decompression, or a combination thereof.

5. The method of claim 1, wherein XNJ is administered at 20 mL/day in 500 mL saline by intravenous drip once daily for 7 days.

6. The method of claim 1, wherein the XNJ stock solution comprises, per 1,000 mL: Moschus 7.5 g; Radix Curcumae 30 g; Jasmine 30 g; and Borneolum 1 g.

7. The method of claim 1, further comprising measuring serum S100B and serum SOD activity at admission and on postoperative Days 1, 3, 5, and 7, wherein adjunct treatment reduces S100B and preserves or restores SOD relative to OT.

8. The method of claim 1, wherein enrollment, ethics, and trial conduct conform to the Declaration of Helsinki and applicable institutional ethics approvals.

9. The method of claim 1, wherein administration of intravenous XNJ is used when oral administration is impracticable, including coma.

10. The method of claim 1, wherein patients are excluded if they: (i) receive more than 400 mL of blood transfusion during treatment; (ii) undergo a second craniotomy; (iii) are discharged against medical advice or die during the assessment phase; or (iv) have a history of allergy to intravenous XNJ.

11. The method of claim 10, wherein the method does not require demonstrating any vascular-perfusion or hemodynamic-rescue effect as a claim limitation.

12. A method of improving neurological recovery after emergency craniotomy in a moderate-to-severee TBI patient, comprising administering XNJ in an amount effective to improve Glasgow Coma Scale (GCS) score during a 7-day postoperative recovery period.

13. The method of claim 12, wherein the administering is in an amount effective to improve Glasgow Outcome Scale (GOS) and/or Karnofsky Performance Status (KPS) at 30 and/or 90 days after surgery.

14. A method of modulating secondary-injury biomarkers after emergency craniotomy in a moderate-to-severee TBI patient, comprising administering XNJ in an amount effective to (i) reduce serum S100B and/or (ii) preserve or increase serum superoxide dismutase (SOD) activity relative to control.

15. The method of claim 14, wherein venous blood is sampled at admission and on postoperative Days 1, 3, 5, and 7 to quantify serum S100B and serum SOD activity.

16. The method of claim 14, wherein reduction of serum S100B and preservation or recovery of serum SOD activity are coupled with improvement in GCS during hospitalization and improvement in GOS and/or KPS at follow-up.
